SEQUENCE LISTING (16 sequences)

SEQ ID NO: 1 - protein, 539 aa, synthetic construct
MSWKVVIIFSLLITPQHGLKESYLEESCSTITEGYLSVLRTGWYTNVFTLEVGDVENLTCSDGPSLIKTELDLTKSALRELKTVSADQLAREEQIENPRQSRFVLGAIALGVATAAAVTAGVAIAKTIRLESEVTAIKNALKTTNEAVSTLGNGVRVLATAVRELKDFVSKNLTRAINKNKCDIDDLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPIKFPEDQFNVALDQVFENIENSQALVDQSNRILSSAEKGNTGFIIVIILIAVLGSSMILVSIFIIIKKTKKPTGAPPELSGVTNNGFIPHN

SEQ ID NO: 2 - DNA, 1620 bp, synthetic construct
  source 1..1620: mol_type = other DNA
atgtcttggaaagtggtgatcattttttcattgctaataacacctcaacacggtcttaaagagagctacctagaagaatcatgtagcactataactgagggatatcttagtgttctgaggacaggttggtataccaacgtttttacattagaggtgggtgatgtagaaaaccttacatgttctgatggacctagcctaataaaaacagaattagatctgaccaaaagtgcactaagagagctcaaaacagtctctgctgaccaattggcaagagaggaacaaattgagaatcccagacaatctaggtttgttctaggagcaatagcactcggtgttgcaacagcagctgcagtcacagcaggtgttgcaattgccaaaaccatccggcttgagagtgaagtcacagcaattaagaatgccctcaaaacgaccaatgaagcagtatctacattggggaatggagttcgagtgttggcaactgcagtgagagagctgaaagactttgtgagcaagaatttaactcgtgcaatcaacaaaaacaagtgcgacattgatgacctaaaaatggccgttagcttcagtcaattcaacagaaggtttctaaatgttgtgcggcaattttcagacaatgctggaataacaccagcaatatctttggacttaatgacagatgctgaactagccagggccgtttctaacatgccgacatctgcaggacaaataaaattgatgttggagaaccgtgcgatggtgcgaagaaaggggttcggaatcctgataggggtctacgggagctccgtaatttacatggtgcagctgccaatctttggcgttatagacacgccttgctggatagtaaaagcagccccttcttgttccgaaaaaaagggaaactatgcttgcctcttaagagaagaccaagggtggtattgtcagaatgcagggtcaactgtttactacccaaatgagaaagactgtgaaacaagaggagaccatgtcttttgcgacacagcagcgggaattaatgttgctgagcaatcaaaggagtgcaacatcaacatatccactacaaattacccatgcaaagtcagcacaggaagacatcctatcagtatggttgcactgtctcctcttggggctctggttgcttgctacaaaggagtaagctgttccattggcagcaacagagtagggatcatcaagcagctgaacaagggttgctcctatataaccaaccaagatgcagacacagtgacaatagacaacactgtatatcagctaagcaaagttgagggtgaacagcatgttataaaaggcagaccagtgtcaagcagctttgatccaatcaagtttcctgaagatcaattcaatgttgcacttgaccaagtttttgagaacattgaaaacagccaggccttggtagatcaatcaaacagaatcctaagcagtgcagagaaagggaatactggcttcatcattgtaataattctaattgctgtccttggctctagcatgatcctagtgagcatcttcattataatcaagaaaacaaagaaaccaacgggagcacctccagagctgagtggtgtcacaaacaatggcttcataccacataattag

SEQ ID NO: 3 - protein, 551 aa, synthetic construct
MSWKVVIIFSLLITPQHGLKESYLEESCSTITEGYLSVLRTGWYTNVFTLEVGDVENLTCSDGPSLIKTELDLTKSALRELKTVSADQLAREEQIENPRRRRFVLGAIALGVATAAAVTAGVAIAKTIRLESEVTAIKNALKTTNEAVSTLGNGVRVLATAVRELKDFVSKNLTRAINKNKCDIDDLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPIKFPEDQFNVALDQVFENIENSQALVDQSNRILSSAEKGNTGGGGSGYIPEAPRDGQAYVRKDGEWVLLSTFLGRSLEVLFQGPGHHHHHHHHSAWSHPQFEK

SEQ ID NO: 4 - DNA, 1656 bp, synthetic construct
  source 1..1656: mol_type = other DNA
atgagttggaaggtggtgattatcttctccctgctgattacaccacaacatggactgaaagagtcctacttggaggagtcctgtagcaccatcacagagggctacctgtctgtgctgaggacaggctggtacaccaatgtgttcaccttggaggtgggagatgtggagaacctgacttgttctgatggaccatccctgattaagacagaactggacctgaccaagtctgccctgagggaactgaaaacagtgtctgctgaccaacttgccagggaggaacagattgagaacccaaggaggaggaggtttgtgctgggagccattgccctgggagtggctacagcagcagcagtgacagcaggagtggctattgccaagaccatcagattggagtctgaggtgacagccatcaagaatgccctgaaaaccaccaatgaggctgtgagcaccctgggcaatggagtgagggtgctggctacagcagtgagggaactgaaagactttgtgagcaagaacctgaccagggctatcaacaagaacaagtgtgacatcgatgacctgaaaatggctgtgtccttcagccagttcaacaggaggttcctgaatgtggtgagacagttctctgacaatgctggcatcacacctgccatctccctggacctgatgacagatgctgaactggcaagggctgtgagcaatatgccaacctctgctggacaaatcaaactgatgttggagaacagggctatggtgaggaggaagggctttggcatcctgattggagtctatggctcctctgtgatttatatggtccaacttccaatctttggagtgattgacacaccatgttggattgtgaaggctgccccatcctgttctgagaagaagggcaactatgcctgtctgctgagggaggaccagggctggtattgtcagaatgctggcagcacagtctactacccaaatgagaaggactgtgagaccaggggagaccatgtgttctgtgacacagcagcaggcatcaatgtggctgaacagagcaaggagtgtaacatcaacatcagcaccaccaactacccatgtaaggtgagcacaggcagacacccaatcagtatggtggctctgagcccactgggagccctggtggcttgttacaagggagtgtcctgtagcattggcagcaacagggtgggcatcatcaagcaacttaacaagggctgttcctacatcaccaaccaggatgctgacacagtgaccattgacaacacagtctaccaacttagcaaggtggagggagaacagcatgtgattaagggcagacctgtgtcctcctcctttgacccaatcaagtttcctgaggaccagttcaatgtggctctggaccaggtgtttgagaacattgagaacagccaggctctggtggaccagagcaacaggattctgtcctctgctgagaagggcaacacaggaggaggaggctctggctacatccctgaggctccaagggatggacaagcctatgtgaggaaggatggagagtgggtgctgctgagcaccttcctgggcaggtccttggaggtgctgttccagggacctggacaccaccaccaccaccaccaccactctgcctggagccacccacagtttgagaagtaa

SEQ ID NO: 5 - protein, 551 aa, synthetic construct
MSWKVVIIFSLLITPQHGLKESYLEESCSTITEGYLSVLRTGWYTNVFTLEVGDVENLTCSDGPSLIKTELDLTKSALRELKTVSADQLAREEQIENPRRRRFVLGAIALGVATAAAVTAGVAIAKTIRLESEVTAIKNALKTTNEAVSTLGNGVRVLATAVRELKDFVSKNLTRAINKNKCDIPDLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPIKFPEDQFNVALDQVFENIENSQALVDQSNRILSSAEKGNTGGGGSGYIPEAPRDGQAYVRKDGEWVLLSTFLGRSLEVLFQGPGHHHHHHHHSAWSHPQFEK

SEQ ID NO: 6 - DNA, 1656 bp, synthetic construct
  source 1..1656: mol_type = other DNA
atgagttggaaggtggtgattatcttctccctgctgattacaccacaacatggactgaaagagtcctacttggaggagtcctgtagcaccatcacagagggctacctgtctgtgctgaggacaggctggtacaccaatgtgttcaccttggaggtgggagatgtggagaacctgacttgttctgatggaccatccctgattaagacagaactggacctgaccaagtctgccctgagggaactgaaaacagtgtctgctgaccaacttgccagggaggaacagattgagaacccaaggaggaggaggtttgtgctgggagccattgccctgggagtggctacagcagcagcagtgacagcaggagtggctattgccaagaccatcagattggagtctgaggtgacagccatcaagaatgccctgaaaaccaccaatgaggctgtgagcaccctgggcaatggagtgagggtgctggctacagcagtgagggaactgaaagactttgtgagcaagaacctgaccagggctatcaacaagaacaagtgtgacatccctgacctgaaaatggctgtgtccttcagccagttcaacaggaggttcctgaatgtggtgagacagttctctgacaatgctggcatcacacctgccatctccctggacctgatgacagatgctgaactggcaagggctgtgagcaatatgccaacctctgctggacaaatcaaactgatgttggagaacagggctatggtgaggaggaagggctttggcatcctgattggagtctatggctcctctgtgatttatatggtccaacttccaatctttggagtgattgacacaccatgttggattgtgaaggctgccccatcctgttctgagaagaagggcaactatgcctgtctgctgagggaggaccagggctggtattgtcagaatgctggcagcacagtctactacccaaatgagaaggactgtgagaccaggggagaccatgtgttctgtgacacagcagcaggcatcaatgtggctgaacagagcaaggagtgtaacatcaacatcagcaccaccaactacccatgtaaggtgagcacaggcagacacccaatcagtatggtggctctgagcccactgggagccctggtggcttgttacaagggagtgtcctgtagcattggcagcaacagggtgggcatcatcaagcaacttaacaagggctgttcctacatcaccaaccaggatgctgacacagtgaccattgacaacacagtctaccaacttagcaaggtggagggagaacagcatgtgattaagggcagacctgtgtcctcctcctttgacccaatcaagtttcctgaggaccagttcaatgtggctctggaccaggtgtttgagaacattgagaacagccaggctctggtggaccagagcaacaggattctgtcctctgctgagaagggcaacacaggaggaggaggctctggctacatccctgaggctccaagggatggacaagcctatgtgaggaaggatggagagtgggtgctgctgagcaccttcctgggcaggtccttggaggtgctgttccagggacctggacaccaccaccaccaccaccaccactctgcctggagccacccacagtttgagaagtaa

SEQ ID NO: 7 - protein, 551 aa, synthetic construct
MSWKVVIIFSLLITPQHGLKESYLEESCSTITEGYLSVLRTGWYTVVFTLEVGDVENLTCSDGPSLIKTELDLTKSALRELKTVSADQLAREEQIENPRRRRFVLGAIALGVATAAAVTAGVAIAKTIRLESEVTAIKNALKTTNEAVSTLGNGVRVLAFAVRELKDFVSKNLTRAINKNKCDIDDLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPIKFPEDQFNVALDQVFENIENSQALVDQSNRILSSAEKGNTGGGGSGYIPEAPRDGQAYVRKDGEWVLLSTFLGRSLEVLFQGPGHHHHHHHHSAWSHPQFEK

SEQ ID NO: 8 - DNA, 1656 bp, synthetic construct
  source 1..1656: mol_type = other DNA
atgagttggaaggtggtgattatcttctccctgctgattacaccacaacatggactgaaagagtcctacttggaggagtcctgtagcaccatcacagagggctacctgtctgtgctgaggacaggctggtacacagtggtgttcaccttggaggtgggagatgtggagaacctgacttgttctgatggaccatccctgattaagacagaactggacctgaccaagtctgccctgagggaactgaaaacagtgtctgctgaccaacttgccagggaggaacagattgagaacccaaggaggaggaggtttgtgctgggagccattgccctgggagtggctacagcagcagcagtgacagcaggagtggctattgccaagaccatcagattggagtctgaggtgacagccatcaagaatgccctgaaaaccaccaatgaggctgtgagcaccctgggcaatggagtgagggtgctggcttttgctgtgagggaactgaaagactttgtgagcaagaacctgaccagggctatcaacaagaacaagtgtgacattgatgacctgaaaatggctgtgtccttcagccagttcaacaggaggttcctgaatgtggtgagacagttctctgacaatgctggcatcacacctgccatctccctggacctgatgacagatgctgaactggcaagggctgtgagcaatatgccaacctctgctggacaaatcaaactgatgttggagaacagggctatggtgaggaggaagggctttggcatcctgattggagtctatggctcctctgtgatttatatggtccaacttccaatctttggagtgattgacacaccatgttggattgtgaaggctgccccatcctgttctgagaagaagggcaactatgcctgtctgctgagggaggaccagggctggtattgtcagaatgctggcagcacagtctactacccaaatgagaaggactgtgagaccaggggagaccatgtgttctgtgacacagcagcaggcatcaatgtggctgaacagagcaaggagtgtaacatcaacatcagcaccaccaactacccatgtaaggtgagcacaggcagacacccaatcagtatggtggctctgagcccactgggagccctggtggcttgttacaagggagtgtcctgtagcattggcagcaacagggtgggcatcatcaagcaacttaacaagggctgttcctacatcaccaaccaggatgctgacacagtgaccattgacaacacagtctaccaacttagcaaggtggagggagaacagcatgtgattaagggcagacctgtgtcctcctcctttgacccaatcaagtttcctgaggaccagttcaatgtggctctggaccaggtgtttgagaacattgagaacagccaggctctggtggaccagagcaacaggattctgtcctctgctgagaagggcaacacaggaggaggaggctctggctacatccctgaggctccaagggatggacaagcctatgtgaggaaggatggagagtgggtgctgctgagcaccttcctgggcaggtccttggaggtgctgttccagggacctggacaccaccaccaccaccaccaccactctgcctggagccacccacagtttgagaagtaa

SEQ ID NO: 9 - protein, 5 aa, synthetic construct
GGGGS

SEQ ID NO: 10 - protein, 27 aa, synthetic construct
GYIPEAPRDGQAYVRKDGEWVLLSTFL

SEQ ID NO: 11 - protein, 10 aa, synthetic construct
SAWSHPQFEK

SEQ ID NO: 12 - protein, 8 aa, synthetic construct
HHHHHHHH

SEQ ID NO: 13 - protein, 7 aa, synthetic construct
ENPRRRR

SEQ ID NO: 14 - protein, 24 aa, synthetic construct
GFIIVIILIAVLGSSMILVSIFII

SEQ ID NO: 15 - protein, 26 aa, synthetic construct
IKKTKKPTGAPPELSGVTNNGFIPHN

SEQ ID NO: 16 - protein, 6 aa, synthetic construct
HHHHHH

(19) United States
(12) Patent Application Publication
(10) Pub. No.: US 2024/0415952 A1
(43) Pub. Date: Dec. 19, 2024

(54) HUMAN METAPNEUMOVIRUS VIRAL VECTOR-BASED VACCINES

(71) Applicants: SANOFI PASTEUR INC., Swiftwater, PA (US); THE UNITED STATES OF AMERICA, AS REPRESENTED BY THE SECRETARY, DEPARTMENT OF HE, Bethesda, MD (US)

(72) Inventors: Yvonne CHAN, Cambridge, MA (US); Sukanya SASMAL, Cambridge, MA (US); Antonia STUEBLER, Cambridge, MA (US); Michael KISHKO, Cambridge, MA (US); Sophia MUNDLE, Cambridge, MA (US); Linong ZHANG, Cambridge, MA (US); Josh DINAPOLI, Cambridge, MA (US); Judith ALAMARES-SAPUAY, Cambridge, MA (US); Natalie ANOSOVA, Cambridge, MA (US); Sudha CHIVUKULA, Cambridge, MA (US); Hillary DANZ, Cambridge, MA (US); Tod STRUGNELL, Cambridge, MA (US); Rachel GROPPO, Cambridge, MA (US); Peter COLLINS, Bethesda, MD (US); Ursula BUCHHOLZ, Bethesda, MD (US); Shirin MUNIR, Bethesda, MD (US); Bibha DAHAL, Bethesda, MD (US)

(21) Appl. No.: 18/671,660

(22) Filed: May 22, 2024

Related U.S. Application Data
(63) Continuation of application No. PCT/US2022/080588, filed on Nov. 29, 2022.
(60) Provisional application No. 63/284,407, filed on Nov. 30, 2021.

(51) Int. Cl. A61K 39/155; A61K 39/00; A61P 31/14; C12N 7/00; C12N 15/86

(52) U.S. Cl. CPC A61K 39/155; A61P 31/14; C12N 7/00; C12N 15/86; A61K 2039/575; A61K 2039/70; C12N 2760/18322; C12N 2760/18334; C12N 2760/18343; C12N 2760/18634

(57) ABSTRACT

The present disclosure provides a human metapneumovirus (hMPV) vaccine comprising an hMPV F protein antigen, and methods of eliciting an immune response by administering said vaccine.

### RELATED APPLICATIONS

This application is a continuation of International Patent Application No. PCT/US2022/080588, filed Nov. 29, 2022, which claims the benefit of U.S. Provisional Application No. 63/284,407, filed on Nov. 30, 2021, which is incorporated by reference in its entirety for all purposes.

### CRADA STATEMENT

This invention was created in the performance of a Cooperative Research and Development Agreement with the National Institutes of Health, an Agency of the Department of Health and Human Services. The Government of the United States has certain rights in this invention.

### SEQUENCE LISTING

The instant application contains a Sequence Listing which has been submitted electronically in XML format and is hereby incorporated by reference in its entirety. Said XML file, created on May 21, 2024, is named 754083_SA9-325BPCCON_ST26.xml and is 23,646 bytes in size.

### BACKGROUND OF THE DISCLOSURE

Human metapneumovirus (hMPV) is a leading cause of acute respiratory infection, particularly in children, immunocompromised patients, and the elderly. hMPV, which is closely related to avian metapneumovirus subtype C, has circulated for at least 65 years, and nearly every child will be infected with hMPV by the age of 5. However, immunity is incomplete, and re-infections occur throughout adult life. Symptoms are similar to those of other respiratory viral infections, ranging from mild (e.g., cough, rhinorrhea, and fever) to severe (e.g., bronchiolitis and pneumonia).

There are currently no licensed vaccines or therapeutics against hMPV despite a high disease burden. Thus, there exists a need for hMPV vaccines that elicit strong immune responses for potent neutralization of an hMPV infection.

### BRIEF SUMMARY OF THE DISCLOSURE

In one aspect, a viral vector that encodes a human metapneumovirus (hMPV) F polypeptide antigen that lacks a transmembrane domain and lacks a cytoplasmic tail, and comprises a human rhinovirus 3C (HRV-3C) protease cleavage site is provided.

In certain exemplary embodiments, the viral vector comprises a backbone derived from a parainfluenza virus (PIV).

In certain exemplary embodiments, said F polypeptide further comprises an F0 cleavage site mutation comprising amino acid substitutions Q100R and S101R, replacing glutamine at amino acid position 100 of SEQ ID NO: 1 with arginine, and replacing serine at amino acid position 101 of SEQ ID NO: 1 with arginine.

In certain exemplary embodiments, said F polypeptide comprises a signal peptide.

In certain exemplary embodiments, said F polypeptide comprises at least one tag sequence that is optionally a polyhistidine-tag (e.g., a 6×His tag, 8×His tag, etc.) and/or a Strep II tag.

In certain exemplary embodiments, said F polypeptide comprises a foldon domain.

In certain exemplary embodiments, said prefusion F polypeptide comprises an amino acid substitution replacing a wild-type amino acid at position 160 of SEQ ID NO: 1, and an amino acid substitution replacing a wild-type amino acid at position 46 of SEQ ID NO: 1.

In certain exemplary embodiments, the F polypeptide comprises an amino acid substitution replacing threonine at amino acid position 160 of SEQ ID NO: 1, and an amino acid substitution replacing asparagine at amino acid position 46 of SEQ ID NO: 1.

In certain exemplary embodiments, said prefusion F polypeptide comprises an amino acid substitution replacing the amino acid at position 160 with phenylalanine, tryptophan, tyrosine, valine, alanine, isoleucine, or leucine. In certain exemplary embodiments, said prefusion F polypeptide comprises an amino acid substitution replacing the amino acid at position 160 with phenylalanine.

In certain embodiments, said prefusion F polypeptide includes a phenylalanine, tryptophan, tyrosine, valine, alanine, isoleucine, or leucine substitution at position 160 of SEQ ID NO: 1, and/or a valine, alanine, isoleucine, leucine, phenylalanine, tyrosine, or proline substitution at position 46 of SEQ ID NO: 1.

In certain exemplary embodiments, said prefusion F polypeptide comprises an amino acid substitution replacing the amino acid at position 46 with valine, alanine, isoleucine, leucine, phenylalanine, tyrosine, or proline. In certain exemplary embodiments, said prefusion F polypeptide comprises an amino acid substitution replacing the amino acid at position 46 with valine.

In certain exemplary embodiments, a live-attenuated virus comprising the viral vector is provided.

In certain exemplary embodiments, a pharmaceutical composition comprising the viral vector is provided.

In certain exemplary embodiments, the live-attenuated virus or the pharmaceutical composition comprises a vaccine.

In certain exemplary embodiments, a method of eliciting an immune response to hMPV and/or HPIV3 or protecting a subject against hMPV infection and/or HPIV3 infection comprises administering the vaccine to a subject.

In certain exemplary embodiments, the vaccine is co-administered with an adjuvant. In certain exemplary embodiments, the vaccine is administered in combination with an additional vaccine. In certain exemplary embodiments, the additional vaccine is a respiratory syncytial virus (RSV) vaccine or an influenza vaccine.

In certain exemplary embodiments, the subject is human. In certain exemplary embodiments, the human subject is an infant, a toddler, or an older adult.

In certain exemplary embodiments, the vaccine increases the serum concentration of neutralizing antibodies, wherein the subject has pre-existing hMPV immunity.

In certain exemplary embodiments, a vaccine for use in eliciting an immune response to hMPV and HPIV3 or protecting a subject against hMPV infection and HPIV3 infection is provided, comprising administering the viral vector, the live-attenuated virus, or the vaccine to a subject.

In certain exemplary embodiments, the viral vector, the live-attenuated virus, or the vaccine is for use in the manufacture of a medicament for eliciting an immune response to hMPV and HPIV3 or protecting a subject against hMPV infection and HPIV3 infection.

In certain exemplary embodiments, a method of eliciting an immune response in a subject in need thereof is provided, comprising administering to the subject, optionally intramuscularly, intranasally, intravenously, subcutaneously, or intradermally, a prophylactically effective amount of the viral vector, the live-attenuated virus, or the vaccine.

In certain exemplary embodiments, a method of preventing an hMPV infection and an HPIV3 infection or reducing one or more symptoms of an hMPV infection and an HPIV3 infection is provided, comprising administering to a subject, optionally intramuscularly, intranasally, intravenously, subcutaneously, or intradermally, a prophylactically effective amount of the viral vector, the live-attenuated virus, or the vaccine.

In certain exemplary embodiments, a use of the viral vector, the live-attenuated virus, or the vaccine, is provided for the manufacture of a medicament for use in treating a subject in need thereof.

In certain exemplary embodiments, the viral vector, the live-attenuated virus, or the vaccine is for use in treating a subject in need thereof.

In certain exemplary embodiments, a kit comprising a container comprising a single-use or multi-use dosage of the viral vector, the live-attenuated virus, or the vaccine is provided, optionally wherein the container is a vial or a pre-filled syringe or injector.

In certain exemplary embodiments, the viral vector, the live-attenuated virus, or the vaccine comprises an hMPV F nucleic acid sequence having at least 95% identity to SEQ ID NO: 8, or comprises SEQ ID NO: 8.

In another aspect, a viral vector that encodes a human metapneumovirus (hMPV) F polypeptide antigen is provided, wherein said F polypeptide lacks a transmembrane domain and lacks a cytoplasmic tail, and comprises: an F₀ cleavage site mutation comprising amino acid substitutions Q100R and S101R; replacing glutamine at amino acid position 100 of SEQ ID NO: 1 with arginine, and replacing serine at amino acid position 101 of SEQ ID NO: 1 with arginine; a human rhinovirus 3C (HRV-3C) protease cleavage site; a heterologous signal peptide; a polyhistidine-tag (e.g., a 6× His tag, 8×His tag, etc.) and/or a Strep II tag; and a foldon domain.

In another aspect, a viral vector that encodes a human metapneumovirus (hMPV) F polypeptide antigen is provided, wherein said F polypeptide lacks a transmembrane domain and lacks a cytoplasmic tail, and comprises an amino acid substitution replacing threonine at amino acid position 160 of SEQ ID NO: 1, and an amino acid substitution replacing asparagine at amino acid position 46 of SEQ ID NO: 1.

In certain exemplary embodiments, said F polypeptide comprises an amino acid substitution replacing threonine at amino acid position 160 with phenylalanine, tryptophan, tyrosine, valine, alanine, isoleucine, or leucine. In certain exemplary embodiments, said F polypeptide comprises an amino acid substitution T160F replacing threonine at amino acid position 160 with phenylalanine.

In certain exemplary embodiments, said F polypeptide comprises an amino acid substitution replacing asparagine at amino acid position 46 with valine, alanine, isoleucine, leucine, phenylalanine, tyrosine, or proline. In certain exemplary embodiments, said F polypeptide comprises an amino acid substitution N46V replacing asparagine at amino acid position 46 with valine.

In certain embodiments, an hMPV polypeptide includes a substitution at position 160 of SEQ ID NO: 1 and a substitution at position 46 of SEQ ID NO: 1, wherein the substitutions are “stabilizing substitutions” that stabilize the tertiary and/or quaternary structure of an hMPV polypeptide. Stabilizing substitutions include, but are not limited to, substitution of: hydrophobic amino acids (e.g., glycine, alanine, valine, leucine, isoleucine, phenylalanine, tyrosine, tryptophan, proline, and methionine); hydrophilic amino acids (e.g., cysteine, serine, threonine, asparagine, and glutamine); amino acids that forms a disulfide bond (e.g., cysteine); amino acids that form hydrogen bonds (e.g., tryptophan, histidine, tyrosine, and phenylalanine); charged amino acids (e.g., aspartic acid, glutamic acid, arginine, lysine, and histidine), and the like.

In certain exemplary embodiments, said F polypeptide comprises at least 95% sequence identity to SEQ ID NO: 7.

In certain exemplary embodiments, said F polypeptide further comprises an F0 cleavage site mutation comprising amino acid substitutions Q100R and S101R, replacing glutamine at amino acid position 100 of SEQ ID NO: 1 with arginine, and replacing serine at amino acid position 101 of SEQ ID NO: 1 with arginine.

In certain exemplary embodiments, said F polypeptide comprises a signal peptide.

In certain exemplary embodiments, said F polypeptide comprises at least one tag sequence that is optionally a polyhistidine-tag (e.g., a 6×His tag, 8×His tag, etc.) and/or a Strep II tag.

In certain exemplary embodiments, said F polypeptide comprises a foldon domain.

In certain exemplary embodiments, a live-attenuated virus comprising the viral vector is provided.

In certain exemplary embodiments, a pharmaceutical composition comprising the viral vector is provided.

In certain exemplary embodiments, the live-attenuated virus or the pharmaceutical composition comprises a vaccine.

In certain exemplary embodiments, a method of eliciting an immune response to hMPV and/or HPIV3 or protecting a subject against hMPV infection and/or HPIV3 infection comprises administering the vaccine to a subject.

In certain exemplary embodiments, the vaccine is co-administered with an adjuvant. In certain exemplary embodiments, the vaccine is administered in combination with an additional vaccine. In certain exemplary embodiments, the additional vaccine is a respiratory syncytial virus (RSV) vaccine or an influenza vaccine.

In certain exemplary embodiments, the subject is human. In certain exemplary embodiments, the human subject is an infant, a toddler, or an older adult.

In certain exemplary embodiments, the vaccine increases the serum concentration of neutralizing antibodies, wherein the subject has pre-existing hMPV immunity.

In certain exemplary embodiments, a vaccine for use in eliciting an immune response to hMPV and HPIV3 or protecting a subject against hMPV infection and HPIV3 infection is provided, comprising administering the viral vector, the live-attenuated virus, or the vaccine to a subject.

In certain exemplary embodiments, the viral vector, the live-attenuated virus, or the vaccine, is for use in the manufacture of a medicament for eliciting an immune response to hMPV and HPIV3 or protecting a subject against hMPV infection and HPIV3 infection.

In certain exemplary embodiments, a method of eliciting an immune response in a subject in need thereof is provided, comprising administering to the subject, optionally intramuscularly, intranasally, intravenously, subcutaneously, or intradermally, a prophylactically effective amount of the viral vector, the live-attenuated virus, or the vaccine.

In certain exemplary embodiments, a method of preventing an hMPV infection and an HPIV3 infection or reducing one or more symptoms of an hMPV infection and an HPIV3 infection is provided, comprising administering to a subject, optionally intramuscularly, intranasally, intravenously, subcutaneously, or intradermally, a prophylactically effective amount of the viral vector, the live-attenuated virus, or the vaccine.

In certain exemplary embodiments, a use of the viral vector, the live-attenuated virus, or the vaccine, is provided for the manufacture of a medicament for use in treating a subject in need thereof.

In certain exemplary embodiments, the viral vector, the live-attenuated virus, or the vaccine is for use in treating a subject in need thereof.

In certain exemplary embodiments, a kit comprising a container comprising a single-use or multi-use dosage of the viral vector, the live-attenuated virus, or the vaccine is provided, optionally wherein the container is a vial or a pre-filled syringe or injector.

In certain exemplary embodiments, the viral vector, the live-attenuated virus, or the vaccine, wherein the vector comprises an hMPV F nucleic acid sequence having at least 95% identity to SEQ ID NO: 8, or comprises SEQ ID NO: 8.

In another aspect, a viral vector that encodes an antigenic human metapneumovirus (hMPV) prefusion F polypeptide is provided, wherein said prefusion F polypeptide lacks a transmembrane domain and lacks a cytoplasmic tail, and comprises: an amino acid substitution T160F replacing threonine at amino acid position 160 of SEQ ID NO: 1 with phenylalanine, and an amino acid substitution N46V replacing asparagine at amino acid position 46 of SEQ ID NO: 1 with valine; an F₀ cleavage site mutation comprising amino acid substitutions Q100R and S101R; replacing glutamine at amino acid position 100 of SEQ ID NO: 1 with arginine, and replacing serine at amino acid position 101 of SEQ ID NO: 1 with arginine; a human rhinovirus 3C (HRV-3C) protease cleavage site; a signal peptide; a polyhistidine-tag (e.g., a 6×His tag, 8×His tag, etc.) and/or a Strep II tag; and a foldon domain.

In certain exemplary embodiments, the hMPV F is from A strain hMPV.

In certain exemplary embodiments, the hMPV F is A1 subtype or A2 subtype hMPV.

In certain exemplary embodiments, said prefusion F polypeptide comprises at least 95% sequence identity to SEQ ID NO: 3 or comprises SEQ ID NO: 3.

In certain exemplary embodiments, said F polypeptide comprises at least 95% sequence identity to SEQ ID NO: 7.

In certain exemplary embodiments, the F polypeptide is a prefusion F polypeptide.

In certain exemplary embodiments, the viral vector backbone is derived from a parainfluenza virus (PIV).

In certain exemplary embodiments, the F polypeptide comprises amino acid substitution T160F replacing threonine at amino acid position 160 with phenylalanine, and amino acid substitution N46V replacing asparagine at amino acid position 46 with valine.

In certain exemplary embodiments, the F polypeptide comprises SEQ ID NO: 7.

In certain exemplary embodiments, the viral vector comprises a nucleic acid molecule having at least 95% sequence identity to SEQ ID NO: 8. In certain exemplary embodiments, the nucleic acid molecule comprises SEQ ID NO: 8.

In certain exemplary embodiments, the hMPV F polypeptide is a pre-fusion F polypeptide.

In certain exemplary embodiments, the viral vector backbone is a chimeric bovine/human parainfluenza type 3 virus (rB/HPIV3) vector backbone.

In certain exemplary embodiments, the viral vector backbone is a human parainfluenza type 3 virus (HPIV3) vector backbone.

In certain exemplary embodiments, a live-attenuated virus comprising the viral vector is provided.

In certain exemplary embodiments, a pharmaceutical composition comprising the viral vector is provided.

In certain exemplary embodiments, the live-attenuated virus or the pharmaceutical composition comprises a vaccine.

In certain exemplary embodiments, a method of eliciting an immune response to hMPV and/or HPIV3 or protecting a subject against hMPV infection and/or HPIV3 infection comprises administering the vaccine to a subject.

In certain exemplary embodiments, the vaccine is co-administered with an adjuvant. In certain exemplary embodiments, the vaccine is administered in combination with an additional vaccine. In certain exemplary embodiments, the additional vaccine is a respiratory syncytial virus (RSV) vaccine or an influenza vaccine.

In certain exemplary embodiments, the subject is human. In certain exemplary embodiments, the human subject is an infant, a toddler, or an older adult.

In certain exemplary embodiments, the vaccine increases the serum concentration of neutralizing antibodies, wherein the subject has pre-existing hMPV immunity.

In certain exemplary embodiments, a vaccine for use in eliciting an immune response to hMPV and HPIV3 or protecting a subject against hMPV infection and HPIV3 infection is provided, comprising administering the viral vector, the live-attenuated virus, or the vaccine to a subject.

In certain exemplary embodiments, the viral vector, the live-attenuated virus, or the vaccine is for use in the manufacture of a medicament for eliciting an immune response to hMPV and HPIV3 or protecting a subject against hMPV infection and HPIV3 infection.

In certain exemplary embodiments, a method of eliciting an immune response in a subject in need thereof is provided, comprising administering to the subject, optionally intramuscularly, intranasally, intravenously, subcutaneously, or intradermally, a prophylactically effective amount of the viral vector, the live-attenuated virus, or the vaccine.

In certain exemplary embodiments, a method of preventing an hMPV infection and an HPIV3 infection or reducing one or more symptoms of an hMPV infection and an HPIV3 infection is provided, comprising administering to a subject, optionally intramuscularly, intranasally, intravenously, subcutaneously, or intradermally, a prophylactically effective amount of the viral vector, the live-attenuated virus, or the vaccine.

In certain exemplary embodiments, a use of the viral vector, the live-attenuated virus, or the vaccine, is provided for the manufacture of a medicament for use in treating a subject in need thereof.

In certain exemplary embodiments, the viral vector, the live-attenuated virus, or the vaccine is for use in treating a subject in need thereof.

In certain exemplary embodiments, a kit comprising a container comprising a single-use or multi-use dosage of the viral vector, the live-attenuated virus, or the vaccine is provided, optionally wherein the container is a vial or a pre-filled syringe or injector.

In certain exemplary embodiments, the viral vector, the live-attenuated virus, or the vaccine, wherein the vector comprises an hMPV F nucleic acid sequence having at least 95% identity to SEQ ID NO: 8, or comprises SEQ ID NO: 8.

### BRIEF DESCRIPTION OF THE DRAWINGS/FIGURES

The foregoing and other features and advantages of the present disclosure will be more fully understood from the following detailed description of illustrative embodiments taken in conjunction with the accompanying drawings. This patent or application file contains at least one drawing executed in color. Copies of this patent or patent application publication with color drawing(s) will be provided by the Office upon request and payment of the necessary fee.

FIG. 1 schematically depicts the design of a recombinant bovine human parainfluenza virus 3 (B/HPIV3) expressing the human metapneumovirus (HMPV) fusion (F) protein from an added gene. The B/HPIV3 backbone contains the N, P, M, and L genes (as labelled) from bovine parainfluenza virus 3 (BPIV3) and the F and HN (as labelled) from human parainfluenza virus 3 (HPIV3). The gene start and gene end transcription signals are shown as light and dark grey boxes, respectively, flanking each ORF. Eight different HMPV (CAN97-83) F versions were inserted at an AscI site between the N and P genes yielding eight virus constructs. The HMPV F insert was designed to be transcribed as a separate mRNA initiating at the P gene start and terminating at the N gene end.

FIG. 2 depicts the design considerations for the panel of 21 candidate hMPV prefusion F antigens shown as two exemplary constructs, D185P and T160F_N46V. The construct D185P is used as a benchmark reference to gauge the activities of various novel constructs. “(mut)”=shaded “ENPRRRR” amino acid sequences and shaded “P”, shaded “V” and shaded “F” singularamino acids; “linker”=bolded, underlined “GGGGS,” “GRS” and “G” amino acid sequences; “foldon”=underlined “GYIPEAPRDGQAYVRKDGEWVLLSTFL” amino acid sequences; “8×HIS”=shaded “HHHHHHHH” amino acid sequences; “Strepll”=shaded “SAWSHPQFEK” sequences.

FIG. 3 depicts the mouse IgG antibody titer against four of the hMPV prefusion F antigen protein constructs measured at day 0, 21, and 35 (data points listed in order from left to right at each time point as follows): (1) A2-F D185P, (2) A2-F T160F_N46V, (3) A2-F K138F, (4) A2-F G366F_K362F, as well as controls: hMPV (5) A1-F pre-F lot 1, (6) A1-F pre-F lot 2, (7) A1-F post F, and (8) B2 pre-F.

FIG. 4 depicts the mouse hMPV microneutralization antibody titer measured at day 21 and 35 against four of the hMPV prefusion F antigen protein constructs: (1) A2-F D185P, (2) A2-F T160F_N46V, (3) A2-F K138F, (4) A2-F G366F_K362F as well as controls: hMPV (1) A1 pre-F lot 1, (2) A1 pre-F lot 2, (3) A1 post F, and (4) B2 pre-F.

FIG. 5 depicts the SEC-MALS results for the reference A1 proteins, A1-A185P and A1-postF and the A2 protein antigen candidates, A2-T160F_N46V and A2-D185P.

FIG. 6 depicts representative melting curves [at fluorescence emission 330 and 350 nm](top panel), smoothened first derivative curve (middle panel), and light scattering [mAU] (bottom panel) for A1-pre-F as well as A1-post-F as measured by nanoDSF.

FIG. 7 depicts a representative melting curve [at fluorescence emission 330 and 350 nm](top panel) and the smoothened first derivative curve (middle panel), and light scattering [mAU](bottom panel) for protein samples derived from the A2-D185P and A2-T160F_N46V constructs as measured by nanoDSF.

FIGS. 8A-8C depict human IgG antibody titer measured at day 14 collected from supernatant of MIMIC co-cultures treated either with IPOL (a polio vaccine) in a 1:50 dilution or an untreated control (no treatment and no human skeletal muscle cells in coculture, “no antigen (w/o HSK)”) to three Polio strains—Polio 1 (FIG. 8A), Polio 2 (FIG. 8B), and Polio 3 (FIG. 8C).

FIGS. 9A-9C depict human IgG antibody titer measured at day 14 collected from supernatant of MIMIC co-cultures treated with 50 ng/ml RSV Pre-F NP (RSV pre-F protein fused to ferritin nanoparticles) treatment to RSV Pre-F (FIG. 9A) and RSV Post-F (FIG. 9B). FIG. 9C depicts whether the antibodies are functional as measured in an RSV neutralization assay.

FIGS. 10A-10B depict human IgG antibody titer measured at day 14 collected from supernatant of MIMIC co-cultures treated with experimental groups—hMPV pre-F protein (at 100 ng/ml or 500 ng/ml) or hMPV post F antigen protein (100 ng/ml) or control groups—no antigen w/o HSK, RSV Pre-F NP, or IPOL to hMPV pre-F (FIG. 10A) or hMPV post-F antigen (FIG. 10B).

FIG. 11 depicts a hMPV microneutralization antibody titer measured at day 14 using collected supernatant of MIMIC co-cultures treated with hMPV pre-F protein (at 100 ng/ml or 500 ng/ml), hMPV post F antigen protein (100 ng/ml), or no antigen w/o HSK.

FIGS. 12A-12B is microscopy images of Vero (FIG. 12A) and A549 (FIG. 12B) cells in 24-well plates, which were infected with the eight different HMPV (CAN97-83) F versions. Cells were fixed and plaques were immunostained for the PIV3 and HMPV F antigens detected with infra-red dye conjugated antibodies shown as green and red, respectively. Stained plaques are shown for each virus as a superimposed image of PIV3 and HMPV F staining. Plaques appear a first color when HMPV F alone is detected, appear a second color when PIV3 antigens alone are detected, and appear a third color when both HMPV F and PIV3 antigens are detected. The tabulated percentages of B/HPIV3 plaques that appeared the third color in FIG. 12A, indicating HMPV F expression, are shown in FIG. 13.

FIG. 13 is the tabulated percentages of B/HPIV3 plaques from FIGS. 12A-12B, which were positive for HMPV F expression as determined by a dual antigen staining plaque assay.

FIG. 14 depicts the expression of viral proteins during in vitro replication. Vero cells were infected with eight different HMPV (CAN97-83) F versions at an MOI of 3 PFU/cell and after 48 hours cell lysates were prepared for analysis by SDS-PAGE (under reducing and denaturing conditions) and Western blotting. BPIV3 N and P, HPIV3 F and HN, and HMPV F were detected using specific primary antibodies followed by infra-red dye conjugated secondary antibodies. GAPDH detection was included as a loading control. Uninfected Vero cell lysate was included as a mock control.

FIG. 15 is a schematic of the experimental design to analyze the protective efficacy and immunogenicity of the eight different HMPV (CAN97-83) F versions in hamsters. Groups of 6 Golden Syrian hamsters received either a single intranasal (IN) dose of one of the eight B/HPIV3-HMPV F viruses or two doses of one of the HMPV purified proteins F-D185P/Q100R/S101R or F-N46V/T160F given with Alum-85 adjuvant by intramuscular (IM) injection 3 weeks apart. Groups of 6 hamsters immunized by the IN route with B/HPIV3 vector (empty) and wt HMPV CAN97-83 (wt hMPV subgroup A) were included as controls. Sera were collected from all groups at 2 weeks post subunit-vaccine boost and 4 weeks post virus immunization to assess the hMPV neutralizing antibody response. Hamsters were challenged IN with wt HMPV (A) (5×10⁵ PFU); lungs and nasal turbinates were collected 3 days post-challenge to examine the HMPV load by plaque assay.

FIG. 16 shows the serum HMPV-neutralizing antibody response in hamsters immunized IN with B/HPIV3 expressing the eight different HMPV (CAN97-83) F versions, or two doses of one of the HMPV purified proteins described in FIG. 15. Serum samples from immunized hamsters were analyzed for HMPV-neutralizing antibodies by 60% plaque reduction neutralization assays on Vero cells. Data were analyzed by one-way ANOVA and Dunnett's multiple comparisons test. The difference between a vaccine candidate and wt HMPV immunized groups was considered statistically significant at 95% confidence interval (P≤0.05) and is indicated by asterisks (**, P≤0.01).

FIGS. 17A-17B demonstrate the protection of the hamster respiratory tract against infection with a wild-type (wt) HMPV challenge. Immunized hamsters were inoculated intranasally with wt HMPV CAN97-83 at a 5×10⁵ PFU challenge dose. On day 3 post-infection, animals were euthanized followed by collection of nasal turbinates and lungs. Tissues were homogenized and HMPV titers were determined by plaque assay on Vero cells. Results are shown as HMPV PFU per gram of tissue (log₁₀) in nasal turbinates (FIG. 17A) and lungs (FIG. 17A). Data were analyzed by one-way ANOVA and Tukey's multiple comparisons test. Differences between each HMPV immunized group and the HMPV naïve [B/HPIV3 vector (empty)] group were considered significant at P≤0.05 and are indicated by asterisks (*, P≤0.05; **, P≤0.01; ***, P≤0.001; ****, P≤0.0001; or ns, not significant).

### DETAILED DESCRIPTION OF THE DISCLOSURE

The present disclosure is directed to, inter alia, novel hMPV F protein vaccines and methods of vaccination with the same. In certain embodiments, the hMPV F protein is expressed from a recombinant chimeric bovine/human parainfluenza virus 3 (rB/HPIV3) vector (a “rB/HPIV3-hMPV F” vector).

### I. Definitions

Unless otherwise defined herein, scientific and technical terms used in connection with the present invention shall have the meanings that are commonly understood by those of ordinary skill in the art. Exemplary methods and materials are described below, although methods and materials similar or equivalent to those described herein can also be used in the practice or testing of the present invention. In case of conflict, the present specification, including definitions, will control. Generally, nomenclature used in connection with, and techniques of, cell and tissue culture, molecular biology, virology, immunology, microbiology, genetics, analytical chemistry, synthetic organic chemistry, medicinal and pharmaceutical chemistry, protein and nucleic acid chemistry, and hybridization described herein are those well-known and commonly used in the art. Enzymatic reactions and purification techniques are performed according to manufacturer's specifications, as commonly accomplished in the art or as described herein. Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. All publications and other references mentioned herein are incorporated by reference in their entirety. Although a number of documents are cited herein, this citation does not constitute an admission that any of these documents forms part of the common general knowledge in the art.

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary of Biochemistry and Molecular Biology, Revised, 2000, Oxford University Press, may provide one of skill with a general dictionary of many of the terms used in this disclosure.

Units, prefixes, and symbols are denoted in their International System of Units (SI) accepted form. Numeric ranges are inclusive of the numbers defining the range. Unless otherwise indicated, amino acid sequences are written left to right in amino to carboxy orientation. The headings provided herein are not limitations of the various aspects of the disclosure. Accordingly, the terms defined immediately below are more fully defined by reference to the specification in its entirety.

The terms “approximately” or “about” are used herein to mean roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower). In some embodiments, the term indicates deviation from the indicated numerical value by ±10%, 5%, 4%, 3%, 2%, 1%, 0.9%, 0.8%, 0.7%, 0.6%, ±0.5%, 0.4%, 0.3%, 0.2%, 0.1%, 0.05%, or 0.01%. In some embodiments, “about” indicates deviation from the indicated numerical value by ±10%. In some embodiments, “about” indicates deviation from the indicated numerical value by ±5%. In some embodiments, “about” indicates deviation from the indicated numerical value by ±4%. In some embodiments, “about” indicates deviation from the indicated numerical value by ±3%. In some embodiments, “about” indicates deviation from the indicated numerical value by ±2%. In some embodiments, “about” indicates deviation from the indicated numerical value by ±1%. In some embodiments, “about” indicates deviation from the indicated numerical value by ±0.9%. In some embodiments, “about” indicates deviation from the indicated numerical value by ±0.8%. In some embodiments, “about” indicates deviation from the indicated numerical value by ±0.7%. In some embodiments, “about” indicates deviation from the indicated numerical value by ±0.6%. In some embodiments, “about” indicates deviation from the indicated numerical value by ±0.5%. In some embodiments, “about” indicates deviation from the indicated numerical value by ±0.4%. In some embodiments, “about” indicates deviation from the indicated numerical value by ±0.3%. In some embodiments, “about” indicates deviation from the indicated numerical value by ±0.1%. In some embodiments, “about” indicates deviation from the indicated numerical value by ±0.05%. In some embodiments, “about” indicates deviation from the indicated numerical value by ±0.01%.

As used herein, the term “antigen stability” refers to stability of the antigen over time or in solution.

As used herein, the term “cavity filling substitutions” refers to engineered hydrophobic substitutions to fill cavities present in the pre-fusion hMPV F trimer.

As used herein, the terms “F protein” or “hMPV F protein” refer to the protein of hMPV responsible for mediating fusion of the viral envelope and the host cell membrane during viral entry. The F protein may mediate fusion between infected cells and non-infected cells to form multinucleated cells or syncytia.

As used herein, the terms “hMPV F polypeptide” or “F polypeptide” refer to a polypeptide comprising at least one epitope of the hMPV F protein.

As used herein, the term “transmembrane domain” refers to an approximately 23 amino acid sequence near the c-terminus of the hMPV F0/F1 that traverses the membrane of the hMPV virion. In certain embodiments, a transmembrane domain comprises the amino acid sequence GFIIVIILIAVLGSSMILVSIFII of SEQ ID NO: 1.

As used herein, the term “cytoplasmic tail” refers to an approximately 25 amino acid sequence at the c-terminus of the hMPV F0/F1 that is located inside the virion. In certain embodiments, a transmembrane domain comprises the amino acid sequence IKKTKKPTGAPPELSGVTNNGFIPHN of SEQ ID NO: 1.

As used herein, a “foldon domain” refers to a trimerization domain of T4 fibritin.

As used herein, a “signal peptide” or “signal sequence” refers to a peptide of approximately 16-30 amino acids in length present at the amino-terminus or the carboxy-terminus of a polypeptide that functions to translocate the polypeptide to the secretory pathway in the endoplasmic reticulum and the Golgi apparatus. In certain embodiments, a signal sequence corresponds to amino acids 1-18 of any one of SEQ ID NO: 1, 3, 5, and 7.

As used herein a “tag sequence” or “affinity tag” refers to a polypeptide sequence that may be used to purify a polypeptide or a protein comprising the tag sequence. Tag include, for example, polyhistidine-tags (e.g., hexahistidine (6×His tag), octahistidine (8×His tag), etc.), glutathione S-transferase (GST), FLAG, streptavidin-binding peptide (SBP), strep II, maltose-binding protein (MBP), calmodulin-binding protein (CBP), chitin-binding domain (CBD), S protein of RNase A, hemagglutinin (HA), c-Myc, and the like.

As used herein, the term “intra-protomer stabilizing substitutions” refers to amino acid substitutions in hMPV F that stabilize the pre-fusion conformation by stabilizing the interaction within a protomer of the hMPV F trimer.

As used herein, the term “inter-protomer stabilizing substitutions” refers to amino acid substitutions in hMPV F that stabilize the pre-fusion conformation by stabilizing the interaction of the protomers of the hMPV F trimer with each other.

As used herein, the term “protease cleavage” refers to proteolysis (sometimes also referred to as “clipping”) of susceptible residues (e.g., lysine or arginine) at a protease cleavage site of a polypeptide sequence. Protease cleavage sites include viral protease cleavage sites such as, e.g., an hMPV F0 protease cleavage site, a respiratory syncytial virus (RSV) F0 protease cleavage site, and a human rhinovirus 3C (HRV-3C) protease cleavage site.

As used herein, the term “post-fusion” with respect to hMPV F refers to a stable conformation of hMPV F that occurs after merging of viral and host cell membranes.

As used herein, the term “pre-fusion” with respect to hMPV F refers to a conformation of hMPV F that is adopted before virus-cell interaction.

As used herein, the term “protomer” refers to a structural unit of an oligomeric protein. In the case of hMPV F, an individual unit of the hMPV F trimer is a protomer.

As used herein, the term “immune response” refers to a response of a cell of the immune system, such as a B cell, T cell, dendritic cell, macrophage, or polymorphonucleocyte, to a stimulus such as an antigen or vaccine. An immune response can include any cell of the body involved in a host defense response, including, for example, an epithelial cell that secretes an interferon or a cytokine. An immune response includes, but is not limited to, an innate and/or adaptive immune response.

As used herein, a “protective immune response” refers to an immune response that protects a subject from infection (e.g., prevents infection or prevents the development of disease associated with infection). Methods of measuring immune responses include measuring, for example, proliferation and/or activity of lymphocytes (such as B or T cells), secretion of cytokines or chemokines, inflammation, antibody production, and the like.

As used herein, an “antibody response” is an immune response in which antibodies are produced.

As used herein, an “antigen” refers to an agent that elicits an immune response, and/or an agent that is bound by a T cell receptor (e.g., when presented by an MHC molecule) or to an antibody (e.g., produced by a B cell) when exposed or administered to an organism. In some embodiments, an antigen elicits a humoral response (e.g., including production of antigen-specific antibodies) in an organism. Alternatively, or additionally, in some embodiments, an antigen elicits a cellular response (e.g., involving T-cells whose receptors specifically interact with the antigen) in an organism. A particular antigen may elicit an immune response in one or several members of a target organism (e.g., mice, rabbits, primates, humans), but not in all members of the target organism species. In some embodiments, an antigen elicits an immune response in at least about 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% of the members of a target organism species. In some embodiments, an antigen binds to an antibody and/or T cell receptor and may or may not induce a particular physiological response in an organism. In some embodiments, for example, an antigen may bind to an antibody and/or to a T cell receptor in vitro, whether or not such an interaction occurs in vivo. In some embodiments, an antigen reacts with the products of specific humoral or cellular immunity. Antigens include the hMPV polypeptides as described herein.

As used herein, an “adjuvant” refers to a substance or vehicle that enhances the immune response to an antigen. Adjuvants can include, without limitation, a suspension of minerals (e.g., alum, aluminum hydroxide, or phosphate) on which antigen is adsorbed; a water-in-oil or oil-in-water emulsion in which antigen solution is emulsified in mineral oil or in water (e.g., Freund's incomplete adjuvant). Sometimes, killed mycobacteria is included (e.g., Freund's complete adjuvant) to further enhance antigenicity. Immuno-stimulatory oligonucleotides (e.g., a CpG motif) can also be used as adjuvants (for example, see U.S. Pat. Nos. 6,194,388; 6,207,646; 6,214,806; 6,218,371; 6,239,116; 6,339,068; 6,406,705; and 6,429,199). Adjuvants can also include biological molecules, such as Toll-Like Receptor (TLR) agonists and costimulatory molecules.

As used herein, an “antigenic hMPV polypeptide” refers to a polypeptide comprising all or part of an hMPV amino acid sequence of sufficient length that the molecule is antigenic with respect to hMPV.

As used herein, a virus that is “attenuated” or has an “attenuated phenotype” refers to a virus that has decreased virulence compared to a reference virus under similar conditions of infection. Attenuation is typically associated with decreased virus replication as compared to replication of a reference wild-type virus under similar conditions of infection, and thus “attenuation” and “restricted replication” often are used synonymously. In some hosts (typically non-natural hosts, including experimental animals), disease is not evident during infection with a reference virus in question, and restriction of virus replication can be used as a surrogate marker for attenuation. In some embodiments, a disclosed rB/HPIV3-hMPV F vector that is attenuated exhibits at least about 10-fold or greater decrease, such as at least about 100-fold or greater decrease in virus titer in the upper or lower respiratory tract of a mammal compared to non-attenuated, wild-type virus titer in the upper or lower respiratory tract, respectively, of a mammal of the same species under the same conditions of infection. An attenuated rB/HPIV3-hMPV F vector may display different phenotypes including without limitation altered growth, temperature sensitive growth, host range restricted growth, or plaque size alteration.

As used herein, a “gene” of a rB/HPIV3 vector as described herein refers to a portion of the rB/HPIV3 genome encoding an mRNA and typically begins at the upstream (3′) end with a gene-start (GS) signal and ends at the downstream (5′) end with the gene-end (GE) signal. In this context, the term gene also embraces what is referred to as a “translational open reading frame,” or ORF, particularly in the case where a protein, such as C, is expressed from an additional ORF rather than from a unique mRNA. To construct a disclosed rB/HPIV3 vector, one or more genes or genome segments may be deleted, inserted or substituted in whole or in part.

As used herein, a “host cell” refers to cells in which a vector can be propagated and its nucleic acid expressed. The cell may be prokaryotic or eukaryotic. The term also includes any progeny of the subject host cell. It is understood that all progeny may not be identical to the parental cell since there may be mutations that occur during replication. However, such progeny are included when the term “host cell” is used.

As used herein, an “infectious and self-replicating virus” refers to a virus that is capable of entering and replicating in a cultured cell or cell of an animal or human host to produce progeny virus capable of the same activity.

As used herein, an “isolated” biological component has been substantially separated or purified away from other biological components, such as other biological components in which the component naturally occurs, such as other chromosomal and extrachromosomal DNA, RNA, and proteins. Proteins, peptides, nucleic acids, and viruses that have been “isolated” include those purified by standard purification methods. Isolated does not require absolute purity, and can include protein, peptide, nucleic acid, or virus molecules that are at least 50% pure, such as at least 75%, 80%, 90%, 95%, 98%, 99%, or even 99.9% pure.

As used herein, a “linker” refers to a bi-functional molecule that can be used to link two molecules into one contiguous molecule. A non-limiting example of a peptide linker includes glycine-serine linkers.

As used herein, a “nucleic acid molecule” refers to a polymeric form of nucleotides, which may include both sense and anti-sense strands of RNA, cDNA, genomic DNA, and synthetic forms and mixed polymers of the above. A nucleotide refers to a ribonucleotide, deoxynucleotide or a modified form of either type of nucleotide. The term “nucleic acid molecule,” as used herein, is synonymous with “polynucleotide.” A nucleic acid molecule is usually at least 10 bases in length, unless otherwise specified. The term includes single- and double-stranded forms of DNA. A nucleic acid molecule may include either or both naturally occurring and modified nucleotides linked together by naturally occurring and/or non-naturally occurring nucleotide linkages.

As used herein, a first nucleic acid sequence is “operably linked” with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked nucleic acid sequences are contiguous and, where necessary to join two protein-coding regions, in the same reading frame.

As used herein, a “parainfluenza virus” or “PIV” refers to a number of enveloped, non-segmented, negative-sense single-stranded RNA viruses from family Paramyxoviridae that are descriptively grouped together. This includes all of the members of genus Respirovirus (e.g., HPIV1, HPIV3) and a number of members of genus Rubulavirus (e.g., HPIV2, HPIV4, PIV5). PIVs are made up of two structural modules: (1) an internal ribonucleoprotein core, or nucleocapsid, containing the viral genome; and (2) an outer, roughly spherical lipoprotein envelope. The PIV genome is approximately 15,000 nucleotides in length and encodes at least eight polypeptides. These proteins include the nucleocapsid structural protein (NP, NC, or N depending on the genera), the phosphoprotein (P), the matrix protein (M), the fusion glycoprotein (F), the hemagglutinin-neuraminidase glycoprotein (HN), the large polymerase protein (L), and the C and D proteins. The gene order is 3′-N-P-M-F-HN-L-5′, and each gene encodes a separate protein encoding mRNA, with the P gene containing one or more additional open reading frames (ORFs) encoding accessory proteins.

As used herein, a “polypeptide” refers to any chain of amino acids, regardless of length or post-translational modification (e.g., glycosylation or phosphorylation). “Polypeptide” applies to amino acid polymers including naturally occurring amino acid polymers and non-naturally occurring amino acid polymers as well as in which one or more amino acid residue is a non-natural amino acid, for example, an artificial chemical mimetic of a corresponding naturally occurring amino acid. A “residue” refers to an amino acid or amino acid mimetic incorporated in a polypeptide by an amide bond or amide bond mimetic. A polypeptide has an amino terminal (N-terminal) end and a carboxy terminal (C-terminal) end. “Polypeptide” is used interchangeably with peptide or protein, and is used herein to refer to a polymer of amino acid residues.

As used herein, a “recombinant” nucleic acid molecule or protein is one that has a sequence that is not naturally occurring, for example, includes one or more nucleic acid substitutions, deletions or insertions, and/or has a sequence that is made by an artificial combination of two otherwise separated segments of sequence. This artificial combination can be accomplished, for example, by chemical synthesis, targeted mutation of a naturally occurring nucleic acid molecule or protein, or by artificial manipulation of isolated segments of nucleic acids, for example, by genetic engineering techniques. A recombinant virus is one that includes a genome that includes a recombinant nucleic acid molecule.

As used herein, a “viral vector” refers to an expression vector that can be used to produce an antigenic hMPV polypeptide, e.g., an antigenic hMPV F polypeptide. Suitable viral vectors include, but are not limited to, viral vectors based on: parainfluenza virus; vaccinia virus; poliovirus; adenovirus (see, e.g., Li et al., Invest Opthalmol V is Sci 35:2543 2549, 1994; Borras et al., Gene Ther 6:515 524, 1999; Li and Davidson, PNAS 92:7700 7704, 1995; Sakamoto et al., H Gene Ther 5:1088 1097, 1999; WO 94/12649, WO 93/03769; WO 93/19191; WO 94/28938; WO 95/11984; and WO 95/00655); adeno-associated virus (see, e.g., Ali et al., Hum Gene Ther 9:8186, 1998, Flannery et al., PNAS 94:6916 6921, 1997; Bennett et al., Invest Opthalmol V is Sci 38:2857 2863, 1997; Jomary et al., Gene Ther 4:683 690, 1997, Rolling et al., Hum Gene Ther 10:641 648, 1999; Ali et al., Hum Mol Genet. 5:591 594, 1996; Srivastava in WO 93/09239, Samulski et al., J. Vir. (1989) 63:3822-3828; Mendelson et al., Virol. (1988) 166:154-165; and Flotte et al., PNAS (1993) 90:10613-10617); SV40; herpes simplex virus; human immunodeficiency virus (see, e.g., Miyoshi et al., PNAS 94:10319 23, 1997; Takahashi et al., J Virol 73:7812 7816, 1999); retroviral vectors (e.g., murine leukemia virus, spleen necrosis virus, and vectors derived from retroviruses such as Rous sarcoma virus, Harvey sarcoma virus, avian leukosis virus, lentivirus, human immunodeficiency virus, myeloproliferative sarcoma virus, and mammary tumor virus); and the like.

Depending on the host/vector system utilized, any of a number of suitable transcription and translation control elements, including constitutive and inducible promoters, transcription enhancer elements, transcription terminators, etc. may be used in the expression vector (see e.g., Bitter et al. (1987) Methods in Enzymology, 153:516-544).

In some embodiments, a nucleotide sequence encoding an antigenic hMPV polypeptide, e.g., an antigenic hMPV F polypeptide, is operably linked to a control element, e.g., a transcriptional control element, such as a promoter. The transcriptional control element may be functional in either a eukaryotic cell (e.g., a mammalian cell) or a prokaryotic cell (e.g., a bacterial or archaeal cell). In some embodiments, a nucleotide sequence encoding an antigenic hMPV polypeptide, e.g., an antigenic hMPV F polypeptide, is operably linked to multiple control elements that allow expression of the nucleotide sequence encoding an antigenic hMPV polypeptide, e.g., an antigenic hMPV F polypeptide, in both prokaryotic and eukaryotic cells.

Non-limiting examples of suitable eukaryotic promoters (promoters functional in a eukaryotic cell) include those from cytomegalovirus (CMV) immediate early, herpes simplex virus (HSV) thymidine kinase, early and late SV40, long terminal repeats (LTRs) from retrovirus, and mouse metallothionein-1. The expression vector may also contain a ribosome binding site for translation initiation and a transcription terminator. The expression vector may also include appropriate sequences for amplifying expression. The expression vector may also include nucleotide sequences encoding protein tags (e.g., 6×His tag, hemagglutinin tag, green fluorescent protein, etc.) that are fused to the site-directed modifying polypeptide, thus resulting in a chimeric polypeptide.

In certain embodiments, an antigenic hMPV polypeptide, e.g., an antigenic hMPV F polypeptide, is expressed in a live attenuated virus. As used herein, a “live attenuated virus” refers to a virus that has reduced virulence compared to a wild-type virus. Live attenuated vaccines tend to help with the production of CD8+ cytotoxic T lymphocytes and T-dependent antibody responses. In comparison to inactivated vaccines, attenuated vaccines produce a stronger and more durable immune response with a quick immunity onset. Attenuated vaccines function by stimulating production of antibodies and memory immune cells in a subject response to the specific pathogen which the vaccine protects against. Live attenuated viruses useful for use as vaccines include, but are not limited to, measles virus, mumps virus, rubella virus, varicella virus, yellow fever virus, influenza virus, smallpox virus, polio virus, rotavirus, parainfluenza virus, Japanese encephalitis virus, varicella virus, herpes zoster virus, and the like. In exemplary embodiments, a live attenuated parainfluenza virus is provided.

As used herein, a “recombinant chimeric bovine/human parainfluenza virus 3” or “rB/HPIV3” refers to a chimeric PIV3 comprising a genome comprising a combination of BPIV3 and HPIV3 genes that together make up the full complement of PIV3 genes in the PIV3 genome (N, P, M, F, HN, and L genes). The disclosed rB/HPIV3 vectors are based on a BPIV3 genome having F and HN genes replaced with the corresponding genes from HPIV3 (one example of which is discussed in Schmidt A C et al., J. Virol. 74:8922-8929, 2000). The structural and functional genetic elements that control gene expression, such as gene start and gene end sequences and genome and anti-genome promoters, are BPIV3 structural and functional genetic elements. The rB/HPIV3 vectors described herein are infectious, self-replicating, and attenuated.

In some embodiments, a heterologous gene encoding hMPV F protein or variant thereof is inserted between the N and P genes of the rB/HPIV3 genome to generate a “rB/HPIV3—hMPV F” vector. The disclosed rB/HPIV3-hMPV F vectors are infectious, self-replicating, and attenuated, and can be used to induce a bivalent immune response to hMPV and HPIV3 in a subject. Particularly suitable rB/HPIV3 vectors are described in U.S. Publication No. US 2021/0145958, incorporated herein by reference in its entirety for all purposes.

Non-limiting examples of methods of generating a recombinant parainfluenza virus (such as a rB/HPIV3) including a heterologous gene, methods of attenuating the viruses (e.g., by recombinant or chemical means), as well as viral sequences and reagents for use in such methods are provided in U.S. Patent Application Publication Nos. 2012/0045471, 2010/0119547, 2009/0263883, 2009/0017517, U.S. Pat. Nos. 7,632,508, 7,622,123, 7,250,171, 7,208,161, 7,201,907, 7,192,593, PCT Pub. No. WO 2016/118642, Liang et al. (J. Virol, 88(8): 4237-4250, 2014), and Tang et al. (J Virol, 77(20):10819-10828, 2003), each of which is incorporated by reference herein. In some embodiments, these methods can be modified as needed using the description provided herein to construct a disclosed rB/HPIV3-hMPV F vector.

The genome of the rB/HPIV3-hMPV F vector can include one or more variations (for example, mutations that cause an amino acid deletion, substitution, or insertion) as long as the resulting rB/HPIV3-hMPV F retains the desired biological function, such as a level of attenuation or immunogenicity. These variations in sequence can be naturally occurring variations or they can be engineered through the use of genetic engineering technique. For parainfluenza virus genomes, care may need to be taken that the nucleotide length of the genome is a multiple of 6 due to hexamer phasing. Otherwise, the genome may not be functional. Calain P and Roux L, J. Virol., 1993, PMID: 8392616; Kolakofsky et al.; J. Virol. 1998; PMID 9444980.

Other mutations involve replacement of the 3′ end of genome with its counterpart from antigenome, which is associated with changes in RNA replication and transcription. In addition, the intergenic regions, e.g., the long and variable noncoding regions at the 3′ and 5′ end of each gene, can be shortened or lengthened or changed in sequence content, and the naturally occurring gene overlap (Skiapodopoulos et al., Virology 272(1): 225-34 (2000); Collins et al., Proc. Natl. Acad. Sci. USA 83:4594-4598 (1986); Collins et al., Proc. Natl. Acad. Sci. USA 84:5134-5138 (1987)) can be removed or changed to a different intergenic region by the methods described herein.

In another embodiment, a sequence surrounding a translational start site (typically including a nucleotide in the −3 position) of a selected viral gene is modified, alone or in combination with introduction of an upstream start codon, to modulate gene expression by specifying up- or down-regulation of translation.

Alternatively, or in combination with other modifications disclosed herein, gene expression can be modulated by altering a transcriptional GS signal of a selected gene(s) of the virus. In additional embodiments, modifications to a transcriptional GE signal can be incorporated into the viral genome.

In addition to the above-described modifications to rB/HPIV3-hMPV F, different or additional modifications to the genome can be made to facilitate manipulations, such as the insertion of unique restriction sites in various intergenic regions (e.g., a unique Asc/site between the N and P genes) or elsewhere. Non-translated gene sequences can be removed to increase capacity for inserting foreign sequences.

Introduction of the foregoing modifications into rB/HPIV3-hMPV F can be achieved by a variety of well-known methods. Examples of such techniques are found in see, e.g., Sambrook et al. (Molecular Cloning: A Laboratory Manual, 4th ed., Cold Spring Harbor, N.Y., 2012) and Ausubel et al. (In Current Protocols in Molecular Biology, John Wiley & Sons, New York, through supplement 104, 2013). Thus, defined mutations can be introduced by conventional techniques (e.g., site-directed mutagenesis) into a cDNA copy of the genome or antigenome. The use of antigenome or genome cDNA sub-fragments to assemble a complete antigenome or genome cDNA has the advantage that each region can be manipulated separately (smaller cDNAs are easier to manipulate than large ones) and then readily assembled into a complete cDNA. Thus, the complete antigenome or genome cDNA, or any sub-fragment thereof, can be used as template for oligonucleotide-directed mutagenesis. A mutated sub-fragment can then be assembled into the complete antigenome or genome cDNA. Mutations can vary from single nucleotide changes to replacement of large cDNA pieces containing one or more genes or genome regions.

The disclosed embodiments of rB/HPIV3-hMPV F are self-replicating, that is they are capable of replicating following infection of an appropriate host cell, and have an attenuated phenotype, for example, when administered to a human subject. In exemplary embodiments, the rB/HPIV3-hMPV F is attenuated about 3- to 500-fold or more in the upper respiratory tract and about 100- to 5000-fold or more in the lower respiratory tract in a mammal compared to control HPIV3. In some embodiments, the level of viral replication in vitro is sufficient to provide for production of virus for use on a widespread scale. In some embodiments, the level of viral replication of attenuated paramyxovirus in vitro is at least 10⁶, at least 107, or at least 10⁸ per n.

In some embodiments, the rB/HPIV3-hMPV F vectors can be produced using the reverse genetics recombinant DNA-based technique (Durbin P A et al., Virology 1997; PMID: 9281512; Collins, et al. 1995. Proc Natl Acad. Sci USA 92:11563-11567). This system allows de novo recovery of infectious virus entirely from cDNA in a qualified cell substrate under defined conditions. Reverse genetics provides a means to introduce predetermined mutations into the rB/HPIV3-hMPV F genome via the cDNA intermediate. Specific attenuating mutations were characterized in preclinical studies and combined to achieve the desired level of attenuation. Derivation of vaccine viruses from cDNA minimizes the risk of contamination with adventitious agents and helps to keep the passage history brief and well documented. Once recovered, the engineered virus strains propagate in the same manner as a biologically derived virus. As a result of passage and amplification, the virus does not contain recombinant DNA from the original recovery.

To propagate rB/HPIV3-hMPV F vectors for immunization and other purposes, a number of cell lines which allow for viral growth may be used. Parainfluenza virus grows in a variety of human and animal cells. Exemplary cell lines for propagating attenuated rB/HPIV3-hMPV F virus for immunization include HEp-2 cells, FRhL-DBS2 cells, LLC-MK2 cells, MRC-5 cells, and Vero cells. Highest virus yields are usually achieved with epithelial cell lines such as Vero cells. Cells are typically inoculated with virus at a multiplicity of infection ranging from about 0.001 to 1.0, or more, and are cultivated under conditions permissive for replication of the virus, e.g., at about 30-37° C. and for about 3-10 days, or as long as necessary for virus to reach an adequate titer. Temperature-sensitive viruses often are grown using 32° C. as the “permissive temperature.” Virus is removed from cell culture and separated from cellular components, typically by standard clarification procedures, e.g., centrifugation, and may be further purified as desired using known procedures.

The rB/HPIV3-hMPV F vectors can be tested in various well known and generally accepted in vitro and in vivo models to confirm adequate attenuation, resistance to phenotypic reversion, and immunogenicity. In in vitro assays, the modified virus is tested for temperature sensitivity of virus replication or “ts phenotype,” and for the small plaque phenotype. Modified virus also may be evaluated in an in vitro human airway epithelium (HAE) model, which appears to provide a means of ranking viruses in the order of their relative attenuation in non-human primates and humans (Zhang et al., 2002 J Virol 76:5654-5666; Schaap-Nutt et al., 2010 Vaccine 28:2788-2798; Ilyushina et al., 2012 J Virol 86:11725-11734). Modified viruses are further tested in animal models of HPIV3 or hMPV infection. A variety of animal models (e.g., murine, cotton rat, and primate) are available.

Immunogenicity of a rB/HPIV3-hMPV F vector can be assessed in an animal model (such as a non-human primate, for example an African green monkey), for example, by determining the number of animals that form antibodies to hMPV and HPIV3 after one immunization and after a second immunization, and by measuring the magnitude of that response. In some embodiments, a rB/HPIV3-hMPV F has sufficient immunogenicity if about 60 to 80% of the animals develop antibodies after the first immunization and about 80 to 100% of the animals develop antibodies after the second immunization. In exemplary embodiments, the immune response protects against infection by both hMPV and HPIV3.

Also provided are isolated polynucleotides comprising or consisting of the genome or antigenome of a disclosed rB/HPIV3-hMPV F vector, vectors comprising the polynucleotides, and host cells comprising the polynucleotides or vectors.

As used herein, a “subject” refers to any member of the animal kingdom. In some embodiments, “subject” refers to humans. In some embodiments, “subject” refers to non-human animals. In some embodiments, subjects include, but are not limited to, mammals, birds, reptiles, amphibians, fish, insects, and/or worms. In certain embodiments, the non-human subject is a mammal (e.g., a rodent, a mouse, a rat, a rabbit, a monkey, a dog, a cat, a sheep, cattle, a primate, and/or a pig). In some embodiments, a subject may be a transgenic animal, genetically engineered animal, and/or a clone. In some embodiments, the terms “individual” or “patient” are used and are intended to be interchangeable with “subject.”

In some embodiments, a “subject” is selected from the group consisting of: a subject aged 65 years old or older, a subject aged 18 to 64 years old (18 to <65 years old), a subject aged 12 years or older, a subject aged 12 to 17 years old (12 to <18 years old), a subject aged 6 to 11 years old (6 to <12 years old), a subject aged 2 to 5 years old (2 to <6 years old), a subject aged 1 to 4 years old (1 to <5 years old), a subject aged 2 months to one year old (2 months to <2 years old), and a subject aged 0 to 2 months old (0 to <3 months old).

In some embodiments, a “subject” is selected from the group consisting of: an older adult (e.g., a senior or elderly adult), an adult, an adolescent, a child, a toddler, and an infant. In some embodiments, a “subject” is selected from the group consisting of: an older adult aged 60 years old or older, an elderly person (e.g., 65 years of age or older), an adult (e.g., 18 to 50 years of age or 18-64 years of age), an adolescent aged 12 to 17 years old (e.g., 12 to <18 years old), a child aged 6 to 11 years old (e.g., 6 to <12 years old), a child aged 2 to 5 years old (e.g., 2 to <6 years old), a toddler aged 1 to 4 years old (e.g., 1 to <5 years old), an infant aged 2 months to 1 year old (e.g., 2 months to <2 years old), a newborn (e.g., 0-27 days of age), and is a preterm newborn infant (e.g., gestational age less than 37 weeks). In some embodiments, a subject is in a pediatric age group as defined by the U.S. FDA: neonate (e.g., birth to less than one month (“NEO”); infant (e.g., age 1 month to less than 2 years (“INF”)); child (e.g., two years to less than 12 years of age (“CHI”)); and adolescent (e.g., ages 12 to less than 17 years (“ADO”)). In some embodiments, a subject is in an older adult in an age group as defined by the U.S. FDA as aged 65 years or older or aged 75 years or older. In particularly exemplary embodiments, a subject is an infant (e.g., age 1 month to less than 2 years), a toddler (e.g., 1 to <5 years old), or an older adult (e.g., aged 60 years or older, 65 years or older, or 75 years or older).

As used herein, the terms “vaccination” or “vaccinate” refer to the administration of a composition intended to generate an immune response, for example, to a disease-causing agent. Vaccination can be administered before, during, and/or after exposure to a disease-causing agent, and/or to the development of one or more symptoms, and in some embodiments, before, during, and/or shortly after exposure to the disease-causing agent. In some embodiments, vaccination includes multiple administrations, appropriately or suitably spaced in time, of a vaccinating composition.

The disclosure describes nucleic acid sequences (e.g., DNA and RNA sequences) and amino acid sequences having a certain degree of identity to a given nucleic acid sequence or amino acid sequence, respectively (e.g., to a reference sequence).

“Sequence identity” between two nucleic acid sequences indicates the percentage of nucleotides that are identical between the sequences. “Sequence identity” between two amino acid sequences indicates the percentage of amino acids that are identical between the sequences.

The terms “% identical,” “% identity,” or similar terms are intended to refer, in particular, to the percentage of nucleotides or amino acids which are identical in an optimal alignment between the sequences to be compared. Said percentage is purely statistical, and the differences between the two sequences may be, but are not necessarily randomly distributed over the entire length of the sequences to be compared. Comparisons of two sequences are usually carried out by comparing said sequences, after optimal alignment, with respect to a segment or “window of comparison,” in order to identify local regions of corresponding sequences. The optimal alignment for a comparison may be carried out manually or with the aid of the local homology algorithm by Smith and Waterman, 1981, Ads App. Math. 2, 482, with the aid of the local homology algorithm by Needleman and Wunsch, 1970, J. Mol. Biol. 48, 443, with the aid of the similarity search algorithm by Pearson and Lipman, 1988, Proc. Natl Acad. Sci. USA 88, 2444, or with the aid of computer programs using said algorithms (GAP, BESTFIT, FASTA, BLAST P, BLAST N, and TFASTA in Wisconsin Genetics Software Package, Genetics Computer Group, 575 Science Drive, Madison, Wis.).

Percentage identity is obtained by determining the number of identical positions at which the sequences to be compared correspond, dividing this number by the number of positions compared (e.g., the number of positions in the reference sequence) and multiplying this result by 100.

In some embodiments, the degree of identity is given for a region which is at least about 50%, at least about 60%, at least about 70%, at least about 80%, at least about 90%, or about 100% of the entire length of the reference sequence. For example, if the reference nucleic acid sequence consists of 200 nucleotides, the degree of identity is given for at least about 100, at least about 120, at least about 140, at least about 160, at least about 180, or about 200 nucleotides, in some embodiments, in continuous nucleotides. In some embodiments, the degree of identity is given for the entire length of the reference sequence.

Nucleic acid sequences or amino acid sequences having a particular degree of identity to a given nucleic acid sequence or amino acid sequence, respectively, may have at least one functional property of said given sequence, e.g., and in some instances, are functionally equivalent to said given sequence. In some embodiments, a nucleic acid sequence or amino acid sequence having a particular degree of identity to a given nucleic acid sequence or amino acid sequence is functionally equivalent to said given sequence.

As used herein, the term “kit” refers to a packaged set of related components, such as one or more compounds or compositions and one or more related materials such as solvents, solutions, buffers, instructions, or desiccants.

### II. hMPV F Polypeptide Antigens

Human metapneumovirus (hMPV) is a negative-sense, single-stranded RNA virus belonging to the Metapneumovirus genus within the Pneumoviridae family. hMPV infects airway epithelial cells in the nose and lung and is the second most common cause, after respiratory syncytial virus (RSV), of lower respiratory infection in young children. hMPV is an enveloped virus with a glycoprotein (G protein), small hydrophobic protein (SH protein), and a fusion protein (F protein) on the virion surface.

As it is an enveloped virus, entry of hMPV into host cells requires the fusion of viral and cellular membranes. Pneumovirus attachment and entry usually requires two viral glycoproteins, the fusion (F) and attachment (G) proteins, and membrane fusion promoted by all pneumovirus fusion glycoproteins that have been examined takes place at a neutral pH. In addition to virus-cell membrane fusion, pneumovirus glycoproteins also promote cell-cell fusion. Multinucleated giant cells, termed syncytia, can be found in cell cultures following infection with a variety of pneumoviruses. Cultured cells infected with hMPV form syncytia, but examination of primary human airway epithelial cells infected with hMPV suggests that syncytium formation by this virus may not be a common in vivo occurrence.

hMPV F is a class I fusion glycoprotein, synthesized as an inactive precursor (F0) that needs to be cleaved to become fusion competent. Proteolytic cleavage generates two disulfide-linked subunits (F2 N-terminal to F1) that assemble into a homotrimer. Cleavage occurs at a monobasic cleavage site immediately upstream of the hydrophobic fusion peptide. Cleavage can be achieved in tissue culture by addition of exogenous trypsin to the medium or by addition of a furin-expression plasmid. However, in vivo other serine proteases, such as TMPRSS2, are thought to be likely more relevant for cleavage. The F trimer is incorporated into the virus particle in a metastable, “prefusion” or “pre-F” conformation. To initiate membrane fusion, hMPV F is activated and undergoes a series of stepwise conformational changes in the F protein that drive membrane fusion and result in hMPV F adopting a highly stable “postfusion” or “post-F” conformation.

In certain exemplary embodiments, proteolytic cleavage of F0 is achieved by co-transfection of a plasmid encoding an hMPV F polypeptide and a plasmid encoding furin at a 4:1 ratio hMPV plasmid:furin plasmid.

Provided herein are antigenic hMPV polypeptides comprising an hMPV F polypeptide. The hMPV F polypeptide may comprise the whole sequence of hMPV F or a portion of hMPV F. In certain embodiments, the portion is the ectodomain.

In some embodiments, the hMPV F polypeptide comprises a sequence having at least 80%, 85%, 90%, 95%, 96%, 97%, 98%, 99%, or 99.5% identity to any one of SEQ ID NOs: 1, 3, 5, and 7.

In some embodiments, the hMPV F polypeptide comprises a modified hMPV F polypeptide having at least 80% identity to the polypeptides of any one of SEQ ID NOs: 1, 3, 5, and 7, wherein the hMPV F polypeptide is antigenic.

In some embodiments, the hMPV F polypeptide comprises only the ectodomain portion of the F protein.

The amino acid sequence of F0 for A2-CAN97-83 is:

(SEQ ID NO: 1)
MSWKVVIIFSLLITPQHGLKESYLEESCSTITEGYLSVLRTGWYTNVFTL
EVGDVENLTCSDGPSLIKTELDLTKSALRELKTVSADQLAREEQIENPRQ
SRFVLGAIALGVATAAAVTAGVAIAKTIRLESEVTAIKNALKTTNEAVST
LGNGVRVLATAVRELKDFVSKNLTRAINKNKCDIDDLKMAVSFSQFNRRF
LNVVRQFSDNAGITPAISLDLMTDAELARAVSNMPTSAGQIKLMLENRAM
VRRKGFGILIGVYGSSVIYMVOLPIFGVIDTPCWIVKAAPSCSEKKGNYA
CLLREDQGWYCQNAGSTVYYPNEKDCETRGDHVFCDTAAGINVAEQSKEC
NINISTTNYPCKVSTGRHPISMVALSPLGALVACYKGVSCSIGSNRVGII
KQLNKGCSYITNQDADTVTIDNTVYQLSKVEGEQHVIKGRPVSSSFDPIK
FPEDQFNVALDQVFENIENSQALVDQSNRILSSAEKGNTGFIIVIILIAV
LGSSMILVSIFIIIKKTKKPTGAPPELSGVTNNGFIPHN.

(Accession AAN52910; Version AAN52910.1; DB Source Accession AY145296.1.) The transmembrane is bolded and underlined, and the cytoplasmic tail is bolded.

The nucleotide sequence of F0 for A2-CAN97-83 is:

(SEQ ID NO: 2)
ATGTCTTGGAAAGTGGTGATCATTTTTTCATTGCTAATAACACCTCAACA
CGGTCTTAAAGAGAGCTACCTAGAAGAATCATGTAGCACTATAACTGAGG
GATATCTTAGTGTTCTGAGGACAGGTTGGTATACCAACGTTTTTACATTA
GAGGTGGGTGATGTAGAAAACCTTACATGTTCTGATGGACCTAGCCTAAT
AAAAACAGAATTAGATCTGACCAAAAGTGCACTAAGAGAGCTCAAAACAG
TCTCTGCTGACCAATTGGCAAGAGAGGAACAAATTGAGAATCCCAGACAA
TCTAGGTTTGTTCTAGGAGCAATAGCACTCGGTGTTGCAACAGCAGCTGC
AGTCACAGCAGGTGTTGCAATTGCCAAAACCATCCGGCTTGAGAGTGAAG
TCACAGCAATTAAGAATGCCCTCAAAACGACCAATGAAGCAGTATCTACA
TTGGGGAATGGAGTTCGAGTGTTGGCAACTGCAGTGAGAGAGCTGAAAGA
CTTTGTGAGCAAGAATTTAACTCGTGCAATCAACAAAAACAAGTGCGACA
TTGATGACCTAAAAATGGCCGTTAGCTTCAGTCAATTCAACAGAAGGTTT
CTAAATGTTGTGCGGCAATTTTCAGACAATGCTGGAATAACACCAGCAAT
ATCTTTGGACTTAATGACAGATGCTGAACTAGCCAGGGCCGTTTCTAACA
TGCCGACATCTGCAGGACAAATAAAATTGATGTTGGAGAACCGTGCGATG
GTGCGAAGAAAGGGGTTCGGAATCCTGATAGGGGTCTACGGGAGCTCCGT
AATTTACATGGTGCAGCTGCCAATCTTTGGCGTTATAGACACGCCTTGCT
GGATAGTAAAAGCAGCCCCTTCTTGTTCCGAAAAAAAGGGAAACTATGCT
TGCCTCTTAAGAGAAGACCAAGGGTGGTATTGTCAGAATGCAGGGTCAAC
TGTTTACTACCCAAATGAGAAAGACTGTGAAACAAGAGGAGACCATGTCT
TTTGCGACACAGCAGCGGGAATTAATGTTGCTGAGCAATCAAAGGAGTGC
AACATCAACATATCCACTACAAATTACCCATGCAAAGTCAGCACAGGAAG
ACATCCTATCAGTATGGTTGCACTGTCTCCTCTTGGGGCTCTGGTTGCTT
GCTACAAAGGAGTAAGCTGTTCCATTGGCAGCAACAGAGTAGGGATCATC
AAGCAGCTGAACAAGGGTTGCTCCTATATAACCAACCAAGATGCAGACAC
AGTGACAATAGACAACACTGTATATCAGCTAAGCAAAGTTGAGGGTGAAC
AGCATGTTATAAAAGGCAGACCAGTGTCAAGCAGCTTTGATCCAATCAAG
TTTCCTGAAGATCAATTCAATGTTGCACTTGACCAAGTTTTTGAGAACAT
TGAAAACAGCCAGGCCTTGGTAGATCAATCAAACAGAATCCTAAGCAGTG
CAGAGAAAGGGAATACTGGCTTCATCATTGTAATAATTCTAATTGCTGTC
CTTGGCTCTAGCATGATCCTAGTGAGCATCTTCATTATAATCAAGAAAAC
AAAGAAACCAACGGGAGCACCTCCAGAGCTGAGTGGTGTCACAAACAATG
GCTTCATACCACATAATTAG

In some embodiments, an epitope of the hMPV F protein that is shared between pre-F and post-F is blocked. Blocking an epitope reduces or eliminates the generation of antibodies against the epitope when an RNA (e.g., mRNA) that encodes for the antigenic hMPV F polypeptide is administered to a subject or when an antigenic hMPV F polypeptide is administered to a subject. This can increase the proportion of antibodies that target an epitope specific to a particular conformation of F, such as the pre-fusion conformation (e.g., antibodies that target site 0 and/or site V). Because F has the pre-fusion conformation in viruses that have not yet entered cells, an increased proportion of antibodies that target pre-F can provide a greater degree of neutralization (e.g., expressed as a neutralizing to binding ratio, as described herein).

The hMPV F polypeptides described herein may have deletions or substitutions relative to the wild-type hMPV F protein (e.g., SEQ ID NO: 1).

For example, in certain embodiments, an hMPV polypeptide: (a) lacks a transmembrane domain and lacks a cytoplasmic tail, and comprises a human rhinovirus 3C (HRV-3C) protease cleavage site; (b) comprises a F₀ cleavage site mutation comprising amino acid substitutions Q100R and S101R relative to SEQ ID NO: 1, replacing glutamine at amino acid position 100 with arginine, and replacing serine at amino acid position 101 with arginine; (c) comprises a heterologous signal peptide; (d) comprises at least one tag sequence that is optionally a polyhistidine-tag (e.g., a 6×His tag, 8×His tag, etc.) and/or a Strep II tag; and/or (e) comprises a foldon domain.

In certain embodiments, an hMPV polypeptide lacks a transmembrane domain and lacks a cytoplasmic tail, and comprises: an F0 cleavage site mutation comprising amino acid substitutions Q100R and S101R relative to SEQ ID NO: 1; replacing glutamine at amino acid position 100 with arginine, and replacing serine at amino acid position 101 with arginine; a human rhinovirus 3C (HRV-3C) protease cleavage site; a heterologous signal peptide; a polyhistidine-tag (e.g., a 6×His tag, 8×His tag, etc.) and/or a Strep II tag; and a foldon domain.

In certain embodiments, an hMPV polypeptide includes a valine, alanine, glycine, isoleucine, leucine, or proline substitution at position 185 of SEQ ID NO: 1.

In certain embodiments, said prefusion F polypeptide includes a phenylalanine, tryptophan, tyrosine, valine, alanine, isoleucine, or leucine substitution at position 160 of SEQ ID NO: 1, and/or a valine, alanine, isoleucine, leucine, phenylalanine, tyrosine, or proline substitution at position 46 of SEQ ID NO: 1.

In certain embodiments, an hMPV polypeptide is from A strain hMPV (e.g., an A1 subtype or an A2 subtype).

In certain embodiments, an amino acid sequence comprising a “backbone” F0 polypeptide sequence is provided, set forth as:

(SEQ ID NO: 3)
MSWKVVIIFSLLITPQHGLKESYLEESCSTITEGYLSVLRTGWYTNVFTL
EVGDVENLTCSDGPSLIKTELDLTKSALRELKTVSADQLAREEQIENPRr
rRFVLGAIALGVATAAAVTAGVAIAKTIRLESEVTAIKNALKTTNEAVST
LGNGVRVLATAVRELKDFVSKNLTRAINKNKCDIDDLKMAVSFSQFNRRF
LNVVRQFSDNAGITPAISLDLMTDAELARAVSNMPTSAGQIKLMLENRAM
VRRKGFGILIGVYGSSVIYMVOLPIFGVIDTPCWIVKAAPSCSEKKGNYA
CLLREDQGWYCQNAGSTVYYPNEKDCETRGDHVFCDTAAGINVAEQSKEC
NINISTTNYPCKVSTGRHPISMVALSPLGALVACYKGVSCSIGSNRVGII
KQLNKGCSYITNQDADTVTIDNTVYQLSKVEGEQHVIKGRPVSSSFDPIK
FPEDQFNVALDQVFENIENSQALVDQSNRILSSAEKGNTggggsgyipea
prdgqayvrkdgewvllstflgrslevlfqgpghhhhhhhhsawshpqfe
k.

In certain embodiments, a nucleotide sequence encoding a “backbone” F0 polypeptide sequence is provided, set forth as:

(SEQ ID NO: 4)
ATGAGTTGGAAGGTGGTGATTATCTTCTCCCTGCTGATTACACCACAACA
TGGACTGAAAGAGTCCTACTTGGAGGAGTCCTGTAGCACCATCACAGAGG
GCTACCTGTCTGTGCTGAGGACAGGCTGGTACACCAATGTGTTCACCTTG
GAGGTGGGAGATGTGGAGAACCTGACTTGTTCTGATGGACCATCCCTGAT
TAAGACAGAACTGGACCTGACCAAGTCTGCCCTGAGGGAACTGAAAACAG
TGTCTGCTGACCAACTTGCCAGGGAGGAACAGATTGAGAACCCAAGGAGG
AGGAGGTTTGTGCTGGGAGCCATTGCCCTGGGAGTGGCTACAGCAGCAGC
AGTGACAGCAGGAGTGGCTATTGCCAAGACCATCAGATTGGAGTCTGAGG
TGACAGCCATCAAGAATGCCCTGAAAACCACCAATGAGGCTGTGAGCACC
CTGGGCAATGGAGTGAGGGTGCTGGCTACAGCAGTGAGGGAACTGAAAGA
CTTTGTGAGCAAGAACCTGACCAGGGCTATCAACAAGAACAAGTGTGACA
TCGATGACCTGAAAATGGCTGTGTCCTTCAGCCAGTTCAACAGGAGGTTC
CTGAATGTGGTGAGACAGTTCTCTGACAATGCTGGCATCACACCTGCCAT
CTCCCTGGACCTGATGACAGATGCTGAACTGGCAAGGGCTGTGAGCAATA
TGCCAACCTCTGCTGGACAAATCAAACTGATGTTGGAGAACAGGGCTATG
GTGAGGAGGAAGGGCTTTGGCATCCTGATTGGAGTCTATGGCTCCTCTGT
GATTTATATGGTCCAACTTCCAATCTTTGGAGTGATTGACACACCATGTT
GGATTGTGAAGGCTGCCCCATCCTGTTCTGAGAAGAAGGGCAACTATGCC
TGTCTGCTGAGGGAGGACCAGGGCTGGTATTGTCAGAATGCTGGCAGCAC
AGTCTACTACCCAAATGAGAAGGACTGTGAGACCAGGGGAGACCATGTGT
TCTGTGACACAGCAGCAGGCATCAATGTGGCTGAACAGAGCAAGGAGTGT
AACATCAACATCAGCACCACCAACTACCCATGTAAGGTGAGCACAGGCAG
ACACCCAATCAGTATGGTGGCTCTGAGCCCACTGGGAGCCCTGGTGGCTT
GTTACAAGGGAGTGTCCTGTAGCATTGGCAGCAACAGGGTGGGCATCATC
AAGCAACTTAACAAGGGCTGTTCCTACATCACCAACCAGGATGCTGACAC
AGTGACCATTGACAACACAGTCTACCAACTTAGCAAGGTGGAGGGAGAAC
AGCATGTGATTAAGGGCAGACCTGTGTCCTCCTCCTTTGACCCAATCAAG
TTTCCTGAGGACCAGTTCAATGTGGCTCTGGACCAGGTGTTTGAGAACAT
TGAGAACAGCCAGGCTCTGGTGGACCAGAGCAACAGGATTCTGTCCTCTG
CTGAGAAGGGCAACACAGGAGGAGGAGGCTCTGGCTACATCCCTGAGGCT
CCAAGGGATGGACAAGCCTATGTGAGGAAGGATGGAGAGTGGGTGCTGCT
GAGCACCTTCCTGGGCAGGTCCTTGGAGGTGCTGTTCCAGGGACCTGGAC
ACCACCACCACCACCACCACCACTCTGCCTGGAGCCACCCACAGTTTGAG
AAGTAA

In certain embodiments, an hMPV polypeptide comprises a “backbone” hMPV sequence set forth as SEQ ID NO: 3, and may optionally contain one or more amino acid substitutions. For example, in certain embodiments, an hMPV polypeptide includes a valine, alanine, glycine, isoleucine, leucine, or proline substitution at position 185 of SEQ ID NO: 3.

In certain embodiments, an hMPV polypeptide includes a phenylalanine, tryptophan, tyrosine, valine, alanine, isoleucine, or leucine substitution at position 160 of SEQ ID NO: 3, and/or a valine, alanine, isoleucine, leucine, phenylalanine, tyrosine, or proline substitution at position 46 of SEQ ID NO: 3.

In certain embodiments, an hMPV polypeptide includes an arginine substitution at one or both of positions 100 and 101 of SEQ ID NO: 3.

In certain embodiments, an hMPV polypeptide has at least 80%, 85%, 90%, 91%, 92%, 93%, 94%, 95%, 96%, 97%, 98%, or 99% sequence identity to SEQ ID NO: 3.

In certain embodiments, an amino acid sequence comprising an hMPV polypeptide sequence is provided, set forth as:

(D185P)
(SEQ ID NO: 5)
MSWKVVIIFSLLITPQHGLKESYLEESCSTITEGYLSVLRTGWYTNVFTL
EVGDVENLTCSDGPSLIKTELDLTKSALRELKTVSADQLAREEQIENPRr
rRFVLGAIALGVATAAAVTAGVAIAKTIRLESEVTAIKNALKTTNEAVST
LGNGVRVLATAVRELKDFVSKNLTRAINKNKCDIpDLKMAVSFSQFNRRF
LNVVRQFSDNAGITPAISLDLMTDAELARAVSNMPTSAGQIKLMLENRAM
VRRKGFGILIGVYGSSVIYMVOLPIFGVIDTPCWIVKAAPSCSEKKGNYA
CLLREDQGWYCQNAGSTVYYPNEKDCETRGDHVFCDTAAGINVAEQSKEC
NINISTTNYPCKVSTGRHPISMVALSPLGALVACYKGVSCSIGSNRVGII
KQLNKGCSYITNQDADTVTIDNTVYQLSKVEGEQHVIKGRPVSSSFDPIK
FPEDQFNVALDQVFENIENSQALVDQSNRILSSAEKGNTggggsgyipea
prdgqayvrkdgewvllstflgrslevlfqgpghhhhhhhhsawshpqfe
k.

(Lower case amino acids denote a linker, foldon motif, linker, HRV-3C cleavage site, linker, 8×-His-tag, and strep-tag II region.)

In certain embodiments, a nucleotide sequence encoding an hMPV polypeptide sequence is provided, set forth as:

(D185P)
(SEQ ID NO: 6)
ATGAGTTGGAAGGTGGTGATTATCTTCTCCCTGCTGATTACACCACAACA
TGGACTGAAAGAGTCCTACTTGGAGGAGTCCTGTAGCACCATCACAGAGG
GCTACCTGTCTGTGCTGAGGACAGGCTGGTACACCAATGTGTTCACCTTG
GAGGTGGGAGATGTGGAGAACCTGACTTGTTCTGATGGACCATCCCTGAT
TAAGACAGAACTGGACCTGACCAAGTCTGCCCTGAGGGAACTGAAAACAG
TGTCTGCTGACCAACTTGCCAGGGAGGAACAGATTGAGAACCCAAGGAGG
AGGAGGTTTGTGCTGGGAGCCATTGCCCTGGGAGTGGCTACAGCAGCAGC
AGTGACAGCAGGAGTGGCTATTGCCAAGACCATCAGATTGGAGTCTGAGG
TGACAGCCATCAAGAATGCCCTGAAAACCACCAATGAGGCTGTGAGCACC
CTGGGCAATGGAGTGAGGGTGCTGGCTACAGCAGTGAGGGAACTGAAAGA
CTTTGTGAGCAAGAACCTGACCAGGGCTATCAACAAGAACAAGTGTGACA
TCCCTGACCTGAAAATGGCTGTGTCCTTCAGCCAGTTCAACAGGAGGTTC
CTGAATGTGGTGAGACAGTTCTCTGACAATGCTGGCATCACACCTGCCAT
CTCCCTGGACCTGATGACAGATGCTGAACTGGCAAGGGCTGTGAGCAATA
TGCCAACCTCTGCTGGACAAATCAAACTGATGTTGGAGAACAGGGCTATG
GTGAGGAGGAAGGGCTTTGGCATCCTGATTGGAGTCTATGGCTCCTCTGT
GATTTATATGGTCCAACTTCCAATCTTTGGAGTGATTGACACACCATGTT
GGATTGTGAAGGCTGCCCCATCCTGTTCTGAGAAGAAGGGCAACTATGCC
TGTCTGCTGAGGGAGGACCAGGGCTGGTATTGTCAGAATGCTGGCAGCAC
AGTCTACTACCCAAATGAGAAGGACTGTGAGACCAGGGGAGACCATGTGT
TCTGTGACACAGCAGCAGGCATCAATGTGGCTGAACAGAGCAAGGAGTGT
AACATCAACATCAGCACCACCAACTACCCATGTAAGGTGAGCACAGGCAG
ACACCCAATCAGTATGGTGGCTCTGAGCCCACTGGGAGCCCTGGTGGCTT
GTTACAAGGGAGTGTCCTGTAGCATTGGCAGCAACAGGGTGGGCATCATC
AAGCAACTTAACAAGGGCTGTTCCTACATCACCAACCAGGATGCTGACAC
AGTGACCATTGACAACACAGTCTACCAACTTAGCAAGGTGGAGGGAGAAC
AGCATGTGATTAAGGGCAGACCTGTGTCCTCCTCCTTTGACCCAATCAAG
TTTCCTGAGGACCAGTTCAATGTGGCTCTGGACCAGGTGTTTGAGAACAT
TGAGAACAGCCAGGCTCTGGTGGACCAGAGCAACAGGATTCTGTCCTCTG
CTGAGAAGGGCAACACAGGAGGAGGAGGCTCTGGCTACATCCCTGAGGCT
CCAAGGGATGGACAAGCCTATGTGAGGAAGGATGGAGAGTGGGTGCTGCT
GAGCACCTTCCTGGGCAGGTCCTTGGAGGTGCTGTTCCAGGGACCTGGAC
ACCACCACCACCACCACCACCACTCTGCCTGGAGCCACCCACAGTTTGAG
AAGTAA.

In certain embodiments, an hMPV polypeptide has at least 80%, 85%, 90%, 91%, 92%, 93%, 94%, 95%, 96%, 97%, 98% or 99% sequence identity to SEQ ID NO: 5. In certain embodiments, an hMPV polypeptide comprises SEQ ID NO: 5

In certain embodiments, an amino acid sequence comprising an hMPV polypeptide sequence is provided, set forth as:

(T160F_N46V)
(SEQ ID NO: 7)
MSWKVVIIFSLLITPQHGLKESYLEESCSTITEGYLSVLRTGWYTvVFTL
EVGDVENLTCSDGPSLIKTELDLTKSALRELKTVSADQLAREEQIENPRr
rRFVLGAIALGVATAAAVTAGVAIAKTIRLESEVTAIKNALKTTNEAVST
LGNGVRVLAFAVRELKDFVSKNLTRAINKNKCDIDDLKMAVSFSQFNRRF
LNVVRQFSDNAGITPAISLDLMTDAELARAVSNMPTSAGQIKLMLENRAM
VRRKGFGILIGVYGSSVIYMVQLPIFGVIDTPCWIVKAAPSCSEKKGNYA
CLLREDQGWYCQNAGSTVYYPNEKDCETRGDHVFCDTAAGINVAEQSKEC
NINISTTNYPCKVSTGRHPISMVALSPLGALVACYKGVSCSIGSNRVGII
KQLNKGCSYITNQDADTVTIDNTVYQLSKVEGEQHVIKGRPVSSSFDPIK
FPEDQFNVALDQVFENIENSQALVDQSNRILSSAEKGNTggggsgyipea
prdgqayvrkdgewvllstflgrslevlfqgpghhhhhhhhsawshpqfe
k.

(Lowercase amino acids denote a linker, foldon motif, linker, HRV-3C cleavage site, linker, 8×-His-tag, and strep-tag II region.)

In certain embodiments, a nucleotide sequence encoding an hMPV polypeptide sequence is provided, set forth as:

(T160F_N46V)
(SEQ ID NO: 8)
ATGAGTTGGAAGGTGGTGATTATCTTCTCCCTGCTGATTACACCACAACA
TGGACTGAAAGAGTCCTACTTGGAGGAGTCCTGTAGCACCATCACAGAGG
GCTACCTGTCTGTGCTGAGGACAGGCTGGTACACAGTGGTGTTCACCTTG
GAGGTGGGAGATGTGGAGAACCTGACTTGTTCTGATGGACCATCCCTGAT
TAAGACAGAACTGGACCTGACCAAGTCTGCCCTGAGGGAACTGAAAACAG
TGTCTGCTGACCAACTTGCCAGGGAGGAACAGATTGAGAACCCAAGGAGG
AGGAGGTTTGTGCTGGGAGCCATTGCCCTGGGAGTGGCTACAGCAGCAGC
AGTGACAGCAGGAGTGGCTATTGCCAAGACCATCAGATTGGAGTCTGAGG
TGACAGCCATCAAGAATGCCCTGAAAACCACCAATGAGGCTGTGAGCACC
CTGGGCAATGGAGTGAGGGTGCTGGCTTTTGCTGTGAGGGAACTGAAAGA
CTTTGTGAGCAAGAACCTGACCAGGGCTATCAACAAGAACAAGTGTGACA
TTGATGACCTGAAAATGGCTGTGTCCTTCAGCCAGTTCAACAGGAGGTTC
CTGAATGTGGTGAGACAGTTCTCTGACAATGCTGGCATCACACCTGCCAT
CTCCCTGGACCTGATGACAGATGCTGAACTGGCAAGGGCTGTGAGCAATA
TGCCAACCTCTGCTGGACAAATCAAACTGATGTTGGAGAACAGGGCTATG
GTGAGGAGGAAGGGCTTTGGCATCCTGATTGGAGTCTATGGCTCCTCTGT
GATTTATATGGTCCAACTTCCAATCTTTGGAGTGATTGACACACCATGTT
GGATTGTGAAGGCTGCCCCATCCTGTTCTGAGAAGAAGGGCAACTATGCC
TGTCTGCTGAGGGAGGACCAGGGCTGGTATTGTCAGAATGCTGGCAGCAC
AGTCTACTACCCAAATGAGAAGGACTGTGAGACCAGGGGAGACCATGTGT
TCTGTGACACAGCAGCAGGCATCAATGTGGCTGAACAGAGCAAGGAGTGT
AACATCAACATCAGCACCACCAACTACCCATGTAAGGTGAGCACAGGCAG
ACACCCAATCAGTATGGTGGCTCTGAGCCCACTGGGAGCCCTGGTGGCTT
GTTACAAGGGAGTGTCCTGTAGCATTGGCAGCAACAGGGTGGGCATCATC
AAGCAACTTAACAAGGGCTGTTCCTACATCACCAACCAGGATGCTGACAC
AGTGACCATTGACAACACAGTCTACCAACTTAGCAAGGTGGAGGGAGAAC
AGCATGTGATTAAGGGCAGACCTGTGTCCTCCTCCTTTGACCCAATCAAG
TTTCCTGAGGACCAGTTCAATGTGGCTCTGGACCAGGTGTTTGAGAACAT
TGAGAACAGCCAGGCTCTGGTGGACCAGAGCAACAGGATTCTGTCCTCTG
CTGAGAAGGGCAACACAGGAGGAGGAGGCTCTGGCTACATCCCTGAGGCT
CCAAGGGATGGACAAGCCTATGTGAGGAAGGATGGAGAGTGGGTGCTGCT
GAGCACCTTCCTGGGCAGGTCCTTGGAGGTGCTGTTCCAGGGACCTGGAC
ACCACCACCACCACCACCACCACTCTGCCTGGAGCCACCCACAGTTTGAG
AAGTAA.

In certain embodiments, an hMPV polypeptide has at least 80%, 85%, 90%, 91%, 92%, 93%, 94%, 95%, 96%, 97%, 98%, or 99% sequence identity to SEQ ID NO: 7. In certain embodiments, an hMPV polypeptide comprises SEQ ID NO: 7.

In general, positions in constructs described herein can be mapped onto a reference sequence, e.g., the wild-type sequence of SEQ ID NO: 1 or the backbone sequence of SEQ ID NO: 3, by pairwise alignment, e.g., using the Needleman-Wunsch algorithm with standard parameters (EBLOSUM62 matrix, Gap penalty 10, gap extension penalty 0.5).

### III. Immunogenic Pharmaceutical Compositions

Immunogenic compositions comprising a disclosed rB/HPIV3-hMPV F vector and a pharmaceutically acceptable carrier are also provided. Such compositions can be administered to a subject by a variety of modes, for example, by an intranasal route. Standard methods for preparing administrable immunogenic compositions are described, for example, in such publications as Remington's Pharmaceutical Sciences, 19th Ed., Mack Publishing Company, Easton, Pa., 1995.

Carriers include, but are not limited to, physiologically balanced culture medium, phosphate buffer saline solution, water, emulsions (e.g., oil/water or water/oil emulsions), various types of wetting agents, cryoprotective additives or stabilizers such as proteins, peptides or hydrolysates (e.g., albumin, gelatin), sugars (e.g., sucrose, lactose, sorbitol), amino acids (e.g., sodium glutamate), or other protective agents. The resulting aqueous solutions may be packaged for use as is or lyophilized. Lyophilized preparations are combined with a sterile solution prior to administration for either single or multiple dosing.

The immunogenic composition can contain a bacteriostat to prevent or minimize degradation during storage, including but not limited to effective concentrations (typically 1% w/v) of benzyl alcohol, phenol, m-cresol, chlorobutanol, methylparaben, and/or propylparaben. A bacteriostat may be contraindicated for some patients; therefore, a lyophilized formulation may be reconstituted in a solution either containing or not containing such a component.

The immunogenic composition can contain as pharmaceutically acceptable vehicles substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, and triethanolamine oleate.

The immunogenic composition may optionally include an adjuvant to enhance the immune response of the host. Suitable adjuvants are, for example, toll-like receptor agonists, alum, AlPO₄, alhydrogel, Lipid-A and derivatives or variants thereof, oil-emulsions, saponins, neutral liposomes, liposomes containing the recombinant virus, and cytokines, non-ionic block copolymers, and chemokines. Non-ionic block polymers containing polyoxyethylene (POE) and polyxylpropylene (POP), such as POE-POP-POE block copolymers, MPL™ (3-O-deacylated monophosphoryl lipid A; Corixa, Hamilton, Ind.) and IL-12, among many other suitable adjuvants well known in the art, may be used as an adjuvant (Newman et al., 1998, Critical Reviews in Therapeutic Drug Carrier Systems 15:89-142). These adjuvants have the advantage in that they help to stimulate the immune system in a non-specific way, thus enhancing the immune response to a pharmaceutical product.

In some instances, it may be desirable to combine the immunogenic composition including the rB/HPIV3-hMPV F, with other pharmaceutical products (e.g., vaccines) which induce protective responses to other viral agents, particularly those causing other childhood illnesses. For example, a composition including a rB/HPIV3-hMPV F as described herein can also include other vaccines recommended by the Advisory Committee on Immunization Practices (ACIP; cdc.gov/vaccines/acip/index.html) for the targeted age group (e.g., infants from approximately one to six months of age). These additional vaccines include, but are not limited to, IN-administered vaccines. As such, a rB/HPIV3-hMPV F as described herein may be administered simultaneously with vaccines against, for example, hepatitis B (HepB), diphtheria, tetanus and pertussis (DTaP), pneumococcal bacteria (PCV), Haemophilus influenzae type b (Hib), polio, influenza and rotavirus.

In some embodiments, the immunogenic composition can be provided in unit dosage form for use to induce an immune response in a subject, for example, to prevent HPIV3 and/or hMPV infection in the subject. A unit dosage form contains a suitable single preselected dosage for administration to a subject, or suitable marked or measured multiples of two or more preselected unit dosages, and/or a metering mechanism for administering the unit dose or multiples thereof.

### IV. Packaging and Use of the Immunogenic Pharmaceutical Compositions

The hMPV vaccines described herein can be formulated or packaged for parenteral (e.g., intramuscular, intradermal, or subcutaneous) administration or nasopharyngeal (e.g., intranasal) administration. In various embodiments, the hMPV vaccines may be formulated or packaged for pulmonary administration. In various embodiments, the hMPV vaccines may be formulated or packaged for intravenous administration. The vaccine compositions may be in the form of an extemporaneous formulation, where the composition is lyophilized and reconstituted with a physiological buffer (e.g., PBS) just before use. The vaccine compositions also may be shipped and provided in the form of an aqueous solution or a frozen aqueous solution and can be directly administered to subjects without reconstitution (after thawing, if previously frozen).

Accordingly, the present disclosure provides an article of manufacture, such as a kit, that provides the hMPV vaccine in a single container or provides the hMPV vaccine in one container (e.g., a first container) and a physiological buffer for reconstitution in another container (e.g., a second container). The container(s) may contain a single-use dosage or multi-use dosage. The container(s) may be pre-treated glass vials or ampules. The article of manufacture may include instructions for use as well.

Methods of administration of a vaccine include, but are not limited to, intradermal, intramuscular, intraperitoneal, intravenous, subcutaneous, intranasal, intra-tracheal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents.

A pharmaceutical composition described herein can be delivered, e.g., intramuscularly, subcutaneously, or intravenously, with a standard needle and syringe, which is optionally prefilled. In addition, a pen delivery device (e.g., an injector (e.g., single-chambered or multi-chambered) or an autoinjector pen) has applications in delivering a pharmaceutical composition described herein. Such a pen delivery device can be reusable or disposable. In some embodiments, the vaccine is provided for use in inhalation and is provided in a pre-filled pump, aerosolizer, or inhaler. In certain embodiments, a prefilled syringe may be utilized for drop-wise administration for intranasal delivery.

The hMPV vaccine disclosed herein may be administered to a subject to induce an immune response directed against the hMPV F protein, wherein an anti-antigen antibody titer in the subject is increased following vaccination relative to an anti-antigen antibody titer in a subject that is not vaccinated with the hMPV vaccine disclosed herein, or relative to an alternative vaccine against hMPV. An “anti-antigen antibody” is a serum antibody that binds specifically to the antigen.

Provided herein are methods of eliciting an immune response in a subject by administering an immunogenic composition containing a disclosed rB/HPIV3-hMPV F to the subject. Upon immunization, the subject responds by producing antibodies specific for one or more of hMPV F protein and HPIV3 HN and F proteins. In addition, innate and cell-mediated immune responses are induced, which can provide antiviral effectors as well as regulating the immune response. As a result of the immunization, the host becomes at least partially or completely immune to HPIV3 and/or hMPV infection, or resistant to developing moderate or severe HPIV3 and/or hMPV disease, particularly of the lower respiratory tract.

The immunogenic compositions containing the rB/HPIV3-hMPV F are administered to a subject susceptible to or otherwise at risk of hMPV and/or HPIV3 infection in an “effective amount” which is sufficient to induce or enhance the individual's immune response capabilities against hMPV and/or HPIV3. The immunogenic composition may be administered by any suitable method, including but not limited to, via injection, aerosol delivery, nasal spray, nasal droplets, oral inoculation, or topical application. In an exemplary embodiment, the attenuated virus is administered according to established human intranasal administration protocols (e.g., as discussed in Karron et al. JID 191:1093-104, 2005). Briefly, adults or children are inoculated intranasally via droplet with an effective amount of the rB/HPIV3-hMPV F, typically in a volume of 0.5 ml of a physiologically acceptable diluent or carrier. This has the advantage of simplicity and safety compared to parenteral immunization with a non-replicating virus. It also provides direct stimulation of local respiratory tract immunity, which plays a major role in resistance to hMPV and HPIV3. Further, this mode of vaccination effectively bypasses the immunosuppressive effects of HPIV3- and hMPV-specific maternally derived serum antibodies, which typically are found in the very young.

The embodiments of rB/HPIV3-hMPV F described herein, and immunogenic compositions thereof, are administered to a subject in an amount effective to induce or enhance an immune response against the HPIV3 and hMPV antigens included in the rB/HPIV3-hMPV F in the subject. An effective amount will allow some growth and proliferation of the virus, in order to produce the desired immune response, but will not produce viral-associated symptoms or illnesses.

A desired immune response is to inhibit subsequent infection with hMPV and/or HPIV3. The hMPV and/or HPIV3 infection does not need to be completely inhibited for the method to be effective. For example, administration of an effective amount of a disclosed rB/HPIV3-hMPV F can decrease subsequent hMPV and/or HPIV3 infection (for example, as measured by infection of cells, or by number or percentage of subjects infected by hMPV and/or HPIV3) by a desired amount, for example by at least 10%, at least 20%, at least 50%, at least 60%, at least 70%, at least 80%, at least 90%, at least 95%, at least 98%, or even at least 100% (prevention of detectable hMPV and/or HPIV3 infection), as compared to a suitable control.

Administration of rB/HPIV3-hMPV F to a subject can elicit the production of an immune response that is protective against serious lower respiratory tract disease, such as pneumonia and bronchiolitis, or croup, when the subject is subsequently infected or re-infected with a wild-type hMPV or HPIV3. While the naturally circulating virus is still capable of causing infection, particularly in the upper respiratory tract, there is a reduced possibility of rhinitis as a result of the immunization and a possible boosting of resistance by subsequent infection by wild-type virus. Following immunization, there are detectable levels of host engendered serum and secretory antibodies which are capable of neutralizing homologous (of the same subgroup) wild-type virus in vitro and in vivo. In many instances the host antibodies will also neutralize wild-type virus of a different, non-vaccine subgroup. To achieve higher levels of cross-protection, for example, against heterologous strains of another subgroup, subjects can be immunized with multiple immunogenic compositions that together comprise rB/HPIV3-hMPV F with genomes encoding hMPV F proteins from at least one predominant strain of both hMPV subgroups A and B.

An immunogenic composition including one or more of the disclosed rB/HPIV3-hMPV F viruses can be used in coordinate (or prime-boost) immunization protocols or combinatorial formulations. It is contemplated that there can be several boosts, and that each boost can be a different disclosed immunogen. It is also contemplated in some examples that the boost may be the same immunogen as another boost, or the prime. In certain embodiments, novel combinatorial immunogenic compositions and coordinate immunization protocols employ separate immunogens or formulations, each directed toward eliciting an anti-viral immune response, such as an immune response to hMPV and HPIV3 proteins. Separate immunogenic compositions that elicit the anti-viral immune response can be combined in a polyvalent immunogenic composition administered to a subject in a single immunization step, or they can be administered separately (in monovalent immunogenic compositions) in a coordinate (or prime-boost) immunization protocol.

The resulting immune response can be characterized by a variety of methods. These include taking samples of nasal washes or sera for analysis of hMPV-specific antibodies, which can be detected by tests including, but not limited to, complement fixation, plaque neutralization, enzyme-linked immunosorbent assay, luciferase-immunoprecipitation assay, and flow cytometry. In addition, immune responses can be detected by assay of cytokines in nasal washes or sera, ELISPOT of immune cells from either source, quantitative RT-PCR or microarray analysis of nasal wash or serum samples, and restimulation of immune cells from nasal washes or serum by re-exposure to viral antigen in vitro and analysis for the production or display of cytokines, surface markers, or other immune correlates measures by flow cytometry or for cytotoxic activity against indicator target cells displaying hMPV antigens. In this regard, individuals are also monitored for signs and symptoms of upper respiratory illness.

In order that this invention may be better understood, the following examples are set forth. These examples are for purposes of illustration only and are not to be construed as limiting the scope of the invention in any manner.

### EXAMPLES

The foregoing description of the specific embodiments will so fully reveal the general nature of the disclosure that others can, by applying knowledge within the skill of the art, readily modify and/or adapt for various applications such specific embodiments, without undue experimentation, without departing from the general concept of the present disclosure. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

### Example 1: Design of Stable B/HPIV3-hMPV F Constructs

A recombinant bovine human parainfluenza virus type 3 (B/HPIV3) was used to express the human metapneumovirus (HMPV) fusion (F) protein. B/HPIV3 is a chimeric virus that consists of bovine PIV3 (Kansas strain; GenBank accession: AF178654) in which the fusion (F) and hemagglutinin neuraminidase (HN) genes have been replaced with those from human PIV3 (JS strain; GenBank accession: Z11575). Schmidt et al. (2001), J Virol 75:4594-603.

In this study, B/HPIV3 was used to express the HMPV F protein (strain CAN97-83, subgroup A, GenBank accession: AY297749) from a gene added at the second gene position, between the BPIV3 N and P genes. This provided a live attenuated intranasal (IN) bivalent vaccine against HPIV3 and HMPV. The very high degree of conservation of the F gene nucleotide sequence and F protein amino acid sequence among all HMPV subgroups and strains (95% amino acid identity) indicates that results with this strain will be applicable to HMPV strains generally. It is also generally the case that when a foreign protein can be successfully expressed from a paramyxovirus (e.g., PIV3), it can be expressed from various gene junctions, as was previously shown with this same B/HPIV3 vector. Liang et al. (2014), J Virol 88: 4237-50.

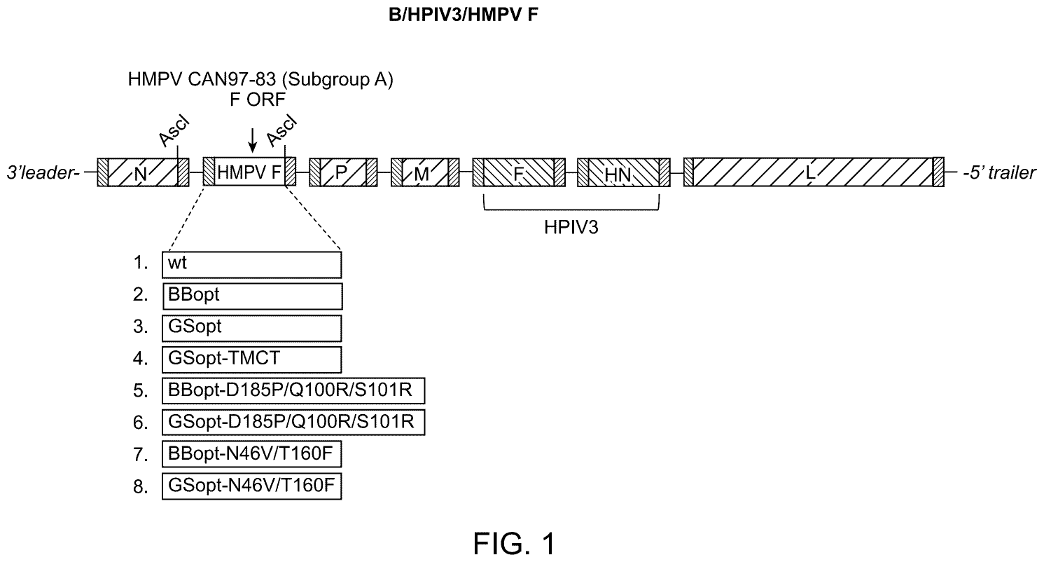

As shown in FIG. 1, eight different versions of the HMPV F protein were constructed to identify forms that were stable and well-accommodated by the vector. Construct 1 expressed the unmodified wild-type form of HMPV F protein of strain CAN97-83. Constructs 2 and 3 expressed forms of this protein that had been codon-optimized (with no change in amino acid coding) by Biobasic, Markham, ON (BBopt; construct 2) or Genscript, Piscataway, NJ (GSopt; construct 3) to increase F expression. Construct 4 (GSopt-TMCT) was a modification of GSopt (construct 3) in which the transmembrane and cytoplasmic tail (TMCT) domains (nt 1474-1620) of the HMPV F ORF were replaced with those of BPIV3 F to potentially enhance packaging into the vector particle. This was based on previous findings where TMCT modification in human respiratory syncytial virus (RSV) F protein significantly enhanced its packaging into the B/HPIV3 vector virions and increased its immunogenicity. Liang et al. (2016), J Virol 90:10022-10038. Constructs 5-8 contained amino acid substitutions (either D185P/Q100R/S101R or N46V/T160F that were introduced into HMPV F to stabilize it in the pre-fusion (pre-F) form. Construct 5 had D185P/Q100R/S101R added to BBopt; construct 6 had D185P/Q100R/S101R added to GSopt; construct 7 had N46V/T160F added to BBopt and construct 8 had N46V/T160F added to GSopt.

Each version of HMPV F was inserted in the B/HPIV3 antigenome at an AscI site located in the downstream non-translated region of the N gene and was designed to enable transcription as a separate mRNA by initiation and termination at the provided P gene-start and N gene-end transcription signals, respectively. The B/HPIV3 vector was used to express eight versions of the F protein of HMPV strain CAN97-83 (subgroup A) from the second gene position, between the BPIV3 N and P genes.

Materials & Methods:

Cells and Viruses. Vero (African green monkey epithelial) and LLC-MK2 (rhesus macaque kidney epithelial) cell lines were cultured in Opti-MEMI medium (Life Technologies, Gaithersburg, MD) containing 5% fetal bovine serum (FBS; Hyclone, Marlborough, MA). The BHK (baby hamster kidney) BSR-T7/5 cell line stably expressing T7 RNA polymerase was maintained as previously described. Buchholz et al. (1999), J Virol 73: 251-259. The culture medium was supplemented with 2% geneticin at every other passage to maintain T7 transgene expression. The human lung epithelial A549 cell line (CCL-185; ATCC, Manassas, VA) was grown in F12 medium (ATCC) containing 5% FBS. The B/HPIV3 P1 and P2 stocks were propagated in LLC-MK2 cells in Opti-MEMI medium containing 2.5% FBS and incubation at 32° C. for 7 days. Wild-type HMPV (Canadian HMPV isolate CAN97-83; subgroup A; GenBank accession: AY297749) was propagated in Vero cells using Opti-MEMI medium containing 2% TrypLE Select (Life Technologies), without FBS, by infecting at an MOI of 0.01 PFU/cell followed by incubation at 32° C. Peret et al. (2002), J Infect Dis 185: 1660-1663. Additional 2% TrypLE Select was added on day 4 and the virus was harvested on day 7. Infected cell supernatants were clarified by centrifugation, snap-frozen on dry ice and stored at −80° C.

Virus rescue. Viruses were recovered by co-transfecting BHK BSR-T7/5 cells with a full-length virus antigenome plasmid together with support plasmids expressing BPIV3 N, P, and L proteins. At 48 hours post-transfection, the transfected cells were co-cultured with a 50% confluent monolayer of LLC-MK2 cells and incubated at 32° C. for 7 days after which the culture supernatant containing the virus was clarified and stored at −80° C. This P1 stock was passaged once more in LLC-MK2 cells by infecting at an MOI of 0.01 PFU/cell to obtain a P2 stock. Viral genome sequences were determined by sequencing uncloned overlapping RT-PCR products derived from viral RNA extracted from the P2 virus stocks.

Stability of HMPV F expression by B/HPIV3 in-vitro. Virus stocks were evaluated for the stability of HMPV F expression by double antigen immunostaining plaque assay detecting HMPV F and B/HPIV3 antigens. Vero and A549 cells were infected with serially diluted viruses, overlaid with medium containing 0.9% methylcellulose, and incubated at 32° C. for 6 days. For staining, the cells were fixed with cold 80% methanol, blocked by incubation for 1 hour with Odyssey blocking buffer (Licor Biosciences, Lincoln NE) at room temperature, and then incubated for 1 hour at room temperature with a mixture of three HMPV F-specific human monoclonal antibodies (Adi15614, MPE33, and MPF5h) each at 1:2500 dilution and a rabbit hyperimmune serum prepared against sucrose-gradient-purified HPIV3 (MS456) at 1:5000 dilution in Odyssey blocking buffer (Licor Biosciences). Next, infra-red dye conjugated secondary antibodies of human and rabbit specificity (Licor Biosciences) were used for detection. Plaques were visualized using the Odyssey infra-red scanner (Licor Biosciences) and were pseudo-colored to appear red and green for HMPV F and HPIV3 antigens, respectively. PFUs appearing yellow on superimposing red and green images indicated the expression of HMPV F protein by the B/HPIV3 vector.

### Results:

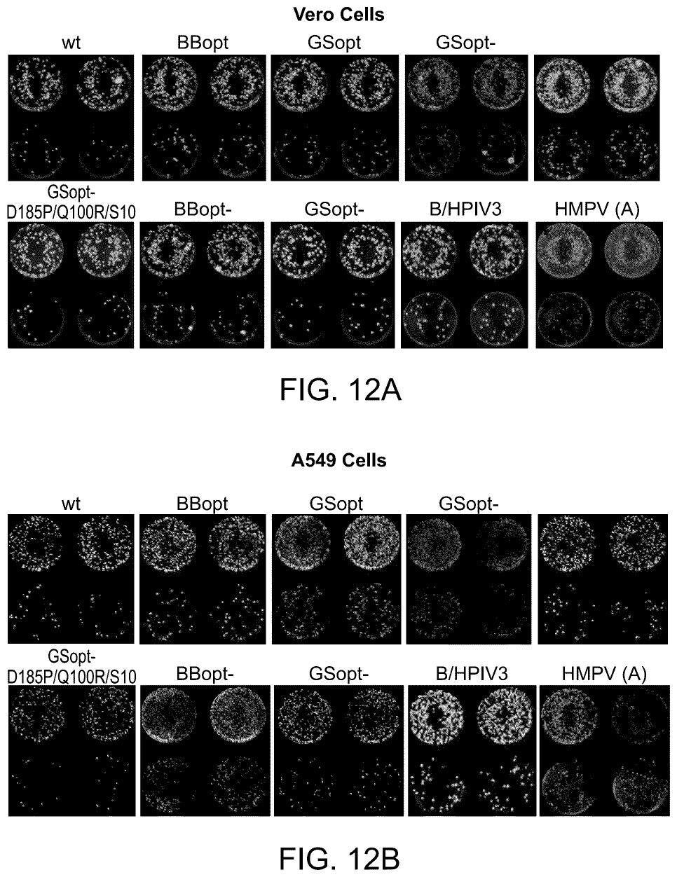

All of the vectors were readily recovered in the transfected BHK BSR-T7/5 cells followed by passage in LLC-MK2 cells. To visualize the plaque phenotype and assess the stability of HMPV F expression, the P1 and P2 virus stocks were subjected to plaque assay on Vero and A549 cells with dual staining for HPIV3 proteins and HMPV F protein. The results for the P2 stock are shown in FIGS. 12A-12B.

Plaques generally were homogeneous in size for each virus but appeared slightly larger in Vero (FIG. 12A) as compared to A549 (FIG. 12B) cells. Without intending to be bound by scientific theory, this could be due to the lack of interferon beta induction in Vero cells as opposed to A549 which are interferon competent. Interestingly, in both A549 and Vero cells, the plaques for all the B/HPIV3 vectors expressing HMPV F were much smaller than the empty vector, suggesting a growth restriction contributed by the HMPV F insert. Furthermore, the virus expressing GSopt-TMCT developed substantially smaller plaques as compared to the other B/HPIV3/HMPV vectors in both Vero and A549 cells (FIGS. 12A-12B), indicating an additional restrictive effect of TMCT mutation on virus growth.

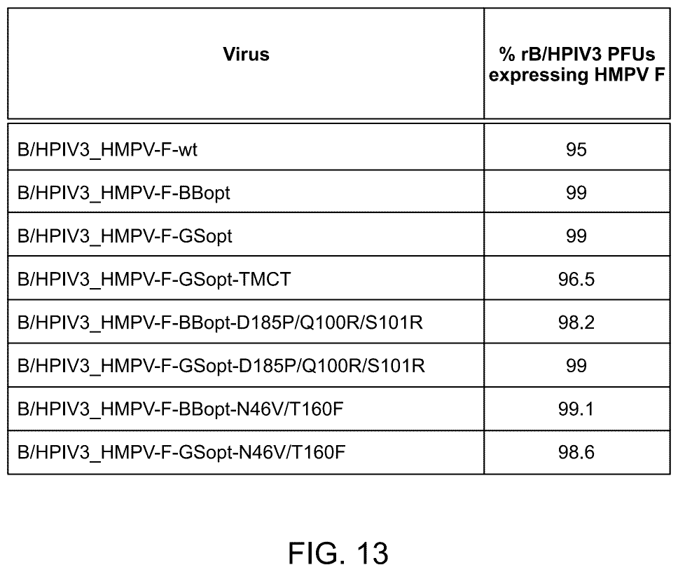

In the dual-antigen staining plaque assay (FIGS. 12A-12B), immunostaining for HMPV F protein was a first color, immunostaining with the HPIV3-specific antiserum was a second color, and plaques expressing both the HMPV F protein and PIV3 proteins were a third color in both Vero and A549 cells (FIGS. 12A-12B). For most of the BHPIV3/HMPV vectors, 95-99% of the plaques from P2 virus stocks (grown in LLC-MK2 cells) were the third color, indicating expression of HMPV F against the background of PIV3 antigens (FIG. 13; derived from data in FIG. 12A). Thus, expression of the insert generally was very stable. This was despite the apparent growth restriction conferred by the added HMPV F gene, which could exert a selective pressure to delete or mutate the added HMPV gene. Instability of expression of a foreign RSV F protein gene by the B/HPIV3 vector had contributed to inadequate immunogenicity in a previous study. Bernstein et al. (2012), Pediatric Infect Dis J 31: 109-114. The one exception was the construct expressing GSopt-TMCT, for which stocks from initial recoveries had substantial proportions of green plaques, indicating loss of expression of HMPV F protein. However, with further recoveries, a P2 stock was identified in which 96% of the plaques were positive for HMPV F protein (FIG. 13). The smaller plaque size and instability of expression for the GSopt-TMCT insert strongly indicated that it was not well-tolerated by the B/HPIV3 vector, such that it inhibited growth and created a negative selective pressure that favored silencing the expression of the insert.

Viral RNA was extracted from P2 stocks for all the vectors and subjected to complete-genome sequencing, which was done by automated sequencing of uncloned overlapping RT-PCR products covering the entire genome except for the 28 and 35 nucleotides at the 3′ and 5′ genome ends, respectively, where the primers bound. No adventitious mutations were identified in any of the viruses. This confirmed the accuracy of the dual-staining plaque assay to detect instability.

### Example 2: Pre-Fusion Stabilized hMPV F Glycoprotein Antigen Constructs

To improve the stability of the prefusion conformation, enhance purification, and induce higher neutralizing antibody titers, a panel of candidate hMPV prefusion F antigen constructs were designed with mutations in the wild-type hMPV-F antigen based on the A2 subtype from Canada designated A2-CAN97-83 (SEQ ID NO: 1).

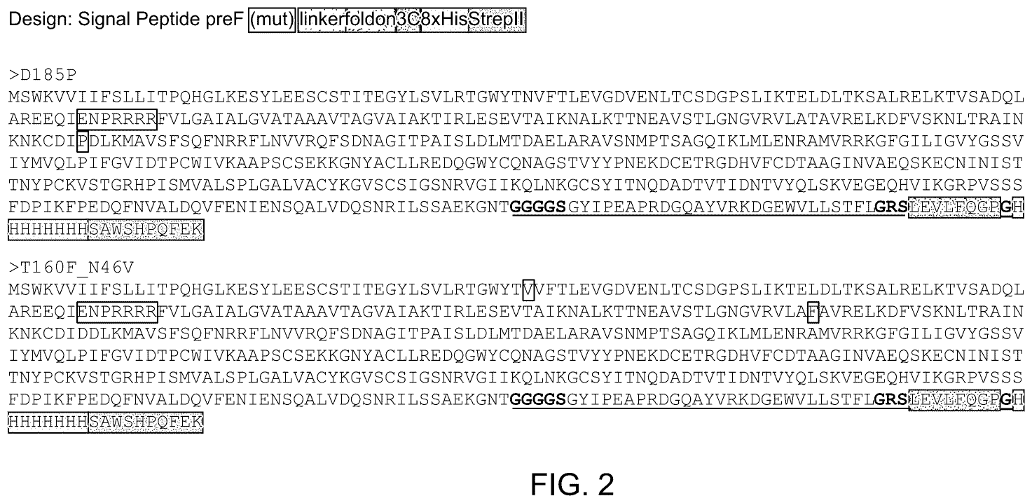

A graphical representation of the design considerations for the panel of candidate hMPV prefusion F antigen constructs are shown in FIG. 2 for two exemplary constructs, D185P (SEQ ID NO: 5) and T160F/N46V (SEQ ID NO: 7). Each construct contained the following characteristics: (1) signal peptide; (2) pre-F cleavage site mutations at amino acid 100-101 (QS to RR); (3) removal of transmembrane domain and cytoplasmic tail; (4) addition of a fibritin motif (i.e., a foldon domain); (5) HRV-3C cleavage site; (6) 8×His tag and Strep II tags; and (7) appropriate linkers for items (4) through (6) (SEQ ID NO: 3).

From this backbone, an in silico analysis was performed to determine single- or double-point mutations that would increase pre-F conformation stability by adding either filling cavity mutations or interface stability mutations. In total, the panel of candidate hMPV prefusion F antigens was comprised of 21 different constructs as shown in column 1 of Table 1.

### Example 3: Evaluation of Protein Expression for the Pre-Fusion Stabilized hMPV F Antigen Constructs

The nucleic acid molecule for each of the candidate hMPV prefusion F antigen constructs was isolated and cloned into an expression vector. Production of protein expression for each construct was evaluated upon mammalian transient transfection using Expi293F human cells. Twenty-four hours after transfection of the constructs, cell lysates or supernatants were recovered for analysis by western blot.

Out of the 21 candidate designs, nine protein antigens were able to be produced. However, only four protein antigens had 90% purity as determined by SDS-PAGE from a 1 L culture. Protein expression characteristics for all of the 21 constructs are shown in Table 1. Constructs that had high protein production and purity had the following mutations: D185P, T160F_N46V, K138F, and G366F_K362F.

TABLE 1
Protein expression characteristics of the 21-
candidate hMPV prefusion F antigen constructs

| ID # | Single or Double Mutations | 1st culture | Purity by SDS-PAGE |
|---|---|---|---|
| 1 | D185P | High | >95% |
| 2 | G366F_K362Y | N/D | |
| 3 | K126F_E431A | N/D | |
| 4 | D87F_E327F | N/D | |
| 5 | R253F_E327F | N/D | |
| 6 | Y425F_A116Y | Low | >70% |
| 7 | Q426F_T119F | Low | >75% |
| 8 | T160F_N46V | High | >95% |
| 9 | K166F_E51F | N/D | |
| 10 | E327F_R329F | N/D | |
| 11 | R304K_D306W | N/D | |
| 12 | T160F_T49Y | N/D | |
| 13 | K126A | N/D | |
| 14 | K138F | High | >90% |
| 15 | T83F_K75F | N/D | |
| 16 | K362L_D454E | Low | >75% |
| 17 | E305F | N/D | |
| 18 | A159F | Low | >60% |
| 19 | G366F_K362F | High | >95% |
| 20 | G366D | Low | >40% |
| 21 | K126F_E431F | N/D | |

### Example 4: Immunogenicity of the Pre-Fusion Stabilized hMPV F Antigen Protein Constructs in Mice

The four candidate hMPV F antigen constructs with 90% purity described in Table 1 were subsequently evaluated for immunogenicity in mice as compared to reference hMPV-F protein from an A1 strain.

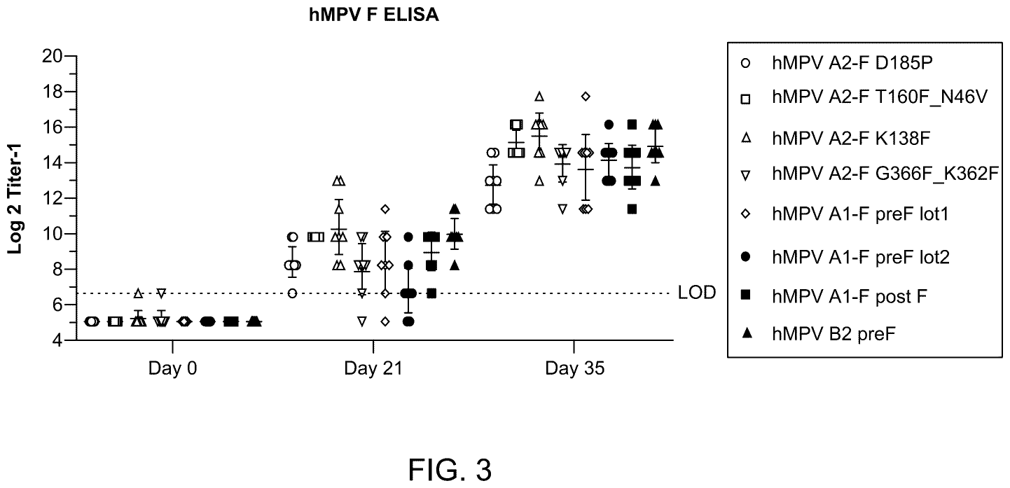

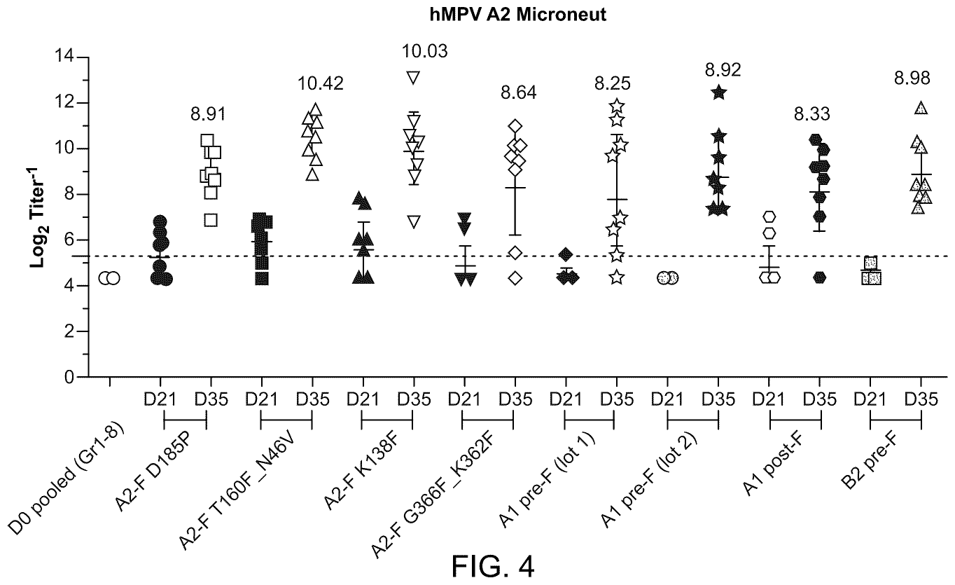

Groups of 8 BALB/c mice (N=8) as shown in Table 2 were administered a 0.5 μg dose of protein antigen adjuvanted with aluminum hydroxide (Al(OH)₃) by intramuscular (IM) injection on day (D) 0 and D2l. All mice were bled and sera was extracted prior to each vaccine administration as well as at two weeks post-last vaccination (D35). Sera was then used to determine the circulating anti-hMPV-F IgG titers as measured by enzyme-linked immunosorbent assay (ELISA) (FIG. 3) and by hMPV microneutralization assay (FIG. 4) to determine the neutralizing activity of the antibody responses. To ensure that all proteins in the post-F group were indeed in the post-F conformation, the proteins were heated to 70° C. for 10 minutes prior to preparation for administration.

TABLE 2
hMPV F antigen vaccine study design in mice

| | | Protein antigen | | | |
|---|---|---|---|---|---|
| | No. of | plus adjuvant | Dose | | |
| Group | mice | (alum) | (μg) | Administration | Rationale |
| 1 | 8 | hMPV A2-F | 0.5 | 50 μl per leg | Test |
| | | D185P | | (100 μl total) | Construct |
| 2 | 8 | hMPV A2-F | 0.5 | 50 μl per leg | Test |
| | | T160F_N46V | | (100 μl total) | Construct |
| 3 | 8 | hMPV A2-F | 0.5 | 50 μl per leg | Test |
| | | K138F | | (100 μl total) | Construct |
| 4 | 8 | hMPV A2-F | 0.5 | 50 μl per leg | Test |
| | | G366F_K362F | | (100 μl total) | construct |
| 5 | 8 | hMPV A1 | 0.5 | 50 μl per leg | A1 pre-F |
| | | pre-F lot 1 | | (100 μl total) | reference |
| 6 | 8 | hMPV A1 | 0.5 | 50 μl per leg | A1 pre-F |
| | | pre-F lot 2 | | (100 μl total) | reference |
| 7 | 8 | hMPV A1 post-F | 0.5 | 50 μl per leg | A1 post-F |
| | | (heat-treated) | | (100 μl total) | reference |
| 8 | 8 | hMPV B2 pre-F | 0.5 | 50 μl per leg | B2 pre-F |
| | | | | (100 μl total) | reference |

The data shows that the construct with the A2-K138F mutation induced the highest binding antibody titer by hMPV-F ELISA followed by A2-T160F_N46V, A2-G366F_K362F, and finally A2-D185P (FIG. 3). When evaluated by microneutralization using an hMPV A2-GFP virus, A2-T160F_N46V had the highest neutralization titer followed by A2-K138F, A2-D185P, and A2-G366F_K362F (FIG. 4).

Although A2-K138F had the highest binding antibody titer and second highest neutralizing antibody titer, this construct was found to form aggregates in solution, indicating potential improper protein folding, and was thus eliminated from further evaluation. A2-G366F_K362F was also eliminated from further evaluation as it had the second lowest binding antibody titer and the lowest neutralizing antibody titer. Therefore, A2-D185P and A2-T160F_N46V were found to induce the highest quality antibodies, and were chosen for advanced analytic analysis to evaluate purity, size and thermal stability as described in Example 5.

### Example 5: Physicochemical Characterization of the Pre-Fusion Stabilized hMPV F Antigen Constructs

To further characterize the purity, size, and thermal stability of the protein produced from the A2-D185P and A2-T160F_N46V constructs—HP-SEC, SEC-HPLC, SEC-MALS, and nanoDSF analysis was performed.

### Purity and Size

The results for the HP-SEC, SEC-HPLC, and SEC-MALS analysis are summarized below in Table 3.

TABLE 3
Summary of SEC evaluations for the pre-fusion stabilized
hMPV F antigen constructs and controls.

| | A1 | A1 | A2 | A2 |
|---|---|---|---|---|
| | A185P | Post-F | T160F_N46V | D185P |
| HP-SEC Trimer (%) | 98.8 | 100 | 94.7 | 97.1 |
| SEC-HPLC MW (kDa) | 341.4 | 337.2 | 384.0 | 321.7 |
| SEC-MALS MW (kDa) | 224.4 | 282.5 | 266.5 | 224.3 |

Molecular weight (MW) from MALS was determined for trimer peak. Conditions for SEC-HPLC were as follows: TSK 3000SWxl SEC column, Phosphate Buffer (0.2M NaH₂PO₄, 0.1 M Arginine, 1% IPA, pH 6.5), flow rate 0.5 ml/min Conditions for SEC-MALS were as follows: 1.7 mM, 200 Å BEH Protein Column, 50 mM Tris buffer at pH 7.5, flow-rate 0.3 ml/min.

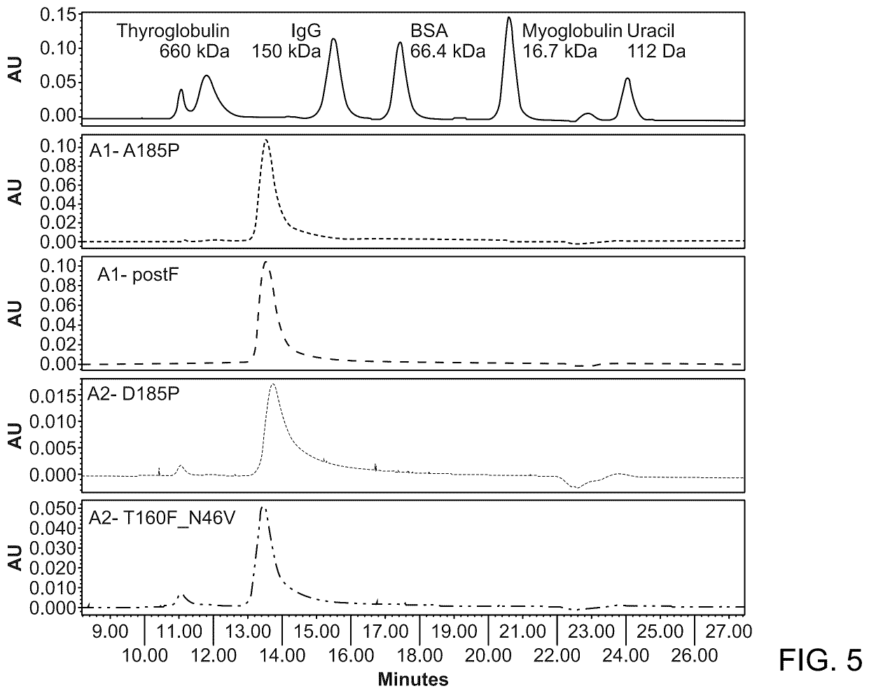

FIG. 5 displays the SEC-MALS results for the reference A1 proteins, A1-A185P and A1-post-F, and below, the A2 protein antigen candidates, A2-T160F_N46V and A2-D185P. Data for all four proteins is also summarized in Table 3. Both A1 reference proteins show ≥98.8% trimer formation and a MW of 224 and 283 kDa for A1-A185P and A1-post-F, respectively. Protein from the A2-T160F_N46V and A2-D185P constructs was composed of 97.4% and 97.1% trimer with a MW of 267 and 224 kDa, respectively.

### Thermal Stability

Onset temperatures (Tonset) and melting points (Tm) of protein denaturation were determined using nano differential scanning fluorimetry (nanoDSF) on both large and small batch lots of A1-Pre-F and A1-Post-F proteins as well as the A2 candidate protein antigens, A2-T160F_N46V and A2-D185P. Samples were diluted in formulation buffer to a final concentration of 0.5 pg/ml and loaded into nanoDSF capillaries in duplicates. All measurements were done using a nanoDSF device. Heating rate was 1.5° C. per minute from 20° C. to 95° C. Data were recorded and analyzed using PR.Stability Analysis v1.01.

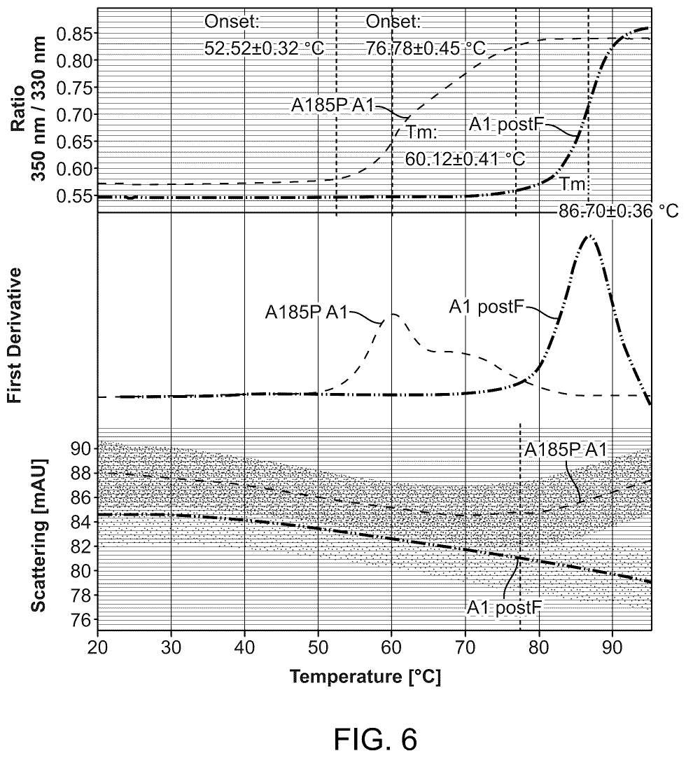

FIG. 6 shows melting curves for A1-preF (A185P) and A2-postF (n=3), yielding Tm values of 60.12° C. and 86.7° C., respectively. This data shows that nanoDSF could differentiate between A1 pre- and post-fusion antigens, with approximately a 27° C. difference in the melting temperature.

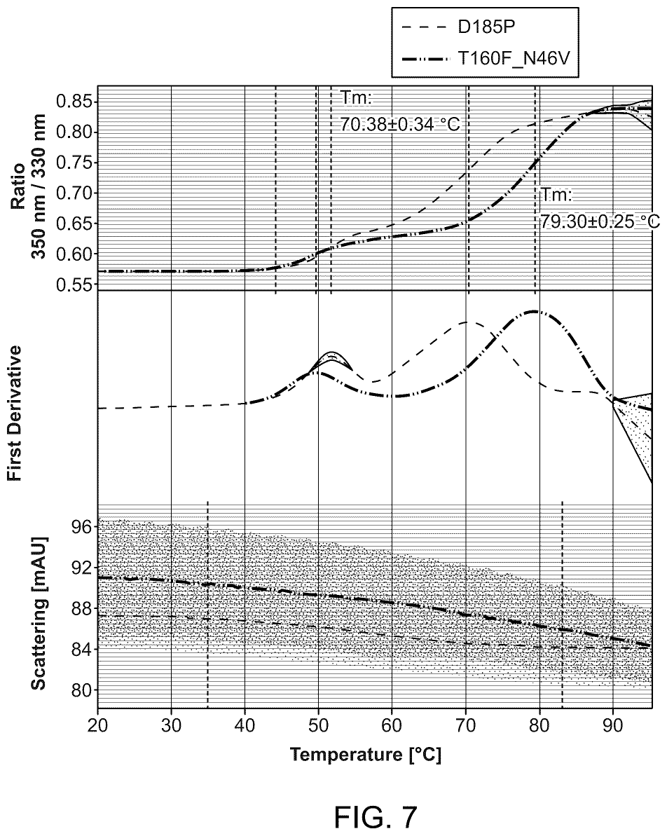

Interestingly, when comparing the thermostability properties of the A2 hMPV-F candidate protein antigens, as seen in FIG. 7, protein derived from the A2-T160F_N46V construct was found to be more thermostable than the more minimally engineered protein produced from the A2-D185P construct, with a melting point increase of nearly 9° C. (Tm 70.4° C. and 79.3° C., respectively).

### Example 6: Immunogenicity of Pre- and Post-Stabilized hMPV F Antigen Protein Constructs in a MIMIC System

### Introduction

The MIMIC© (Modular Immune In vitro Construct) system can stimulate innate and adaptive immune responses in vitro that occur in vaccination/inflection site in vivo. Williams et al. (2015) Sanofi Pasteur poster, “In vitro differentiation of class-switched YF specific antibody secreting cells from naïve B cells.” Using the MIMIC system can recapitulate some aspects unique to human physiology, e.g., HLA haplotypes, age, autoimmune statue, and gender, thereby complementing immunogenicity studies performed in animal models. Higbee et al. (2009) ATLA 37: 19-27.

To this end, pre- and post-hMPV F antigen protein constructs were tested in a MIMIC system to assess the quality of the immunogenic response relative to controls. Control groups included: untreated control (no antigen without human skeletal muscle cells (w/o HSK)), reference antigen—RSV pre-F protein fused to ferritin nanoparticles (pre-F NP) and polio vaccine (IPOL).

### Material & Methods

Briefly, PBMCs were harvested via magnetic bead separation kit from 22 different human blood donors. Human dendritic cells (DCs) and B cells selected therefrom were added to and co-cultured with human skeletal muscle cells (HSKMC) and stimulated with either hMPV pre-F antigen protein (at 100 ng/ml or 500 ng/ml) or hMPV post F antigen protein (100 ng/ml). For B cell responses, following 14-day co-culture, supernatants were collected and analyzed for antibody specificity and function.

### Results:

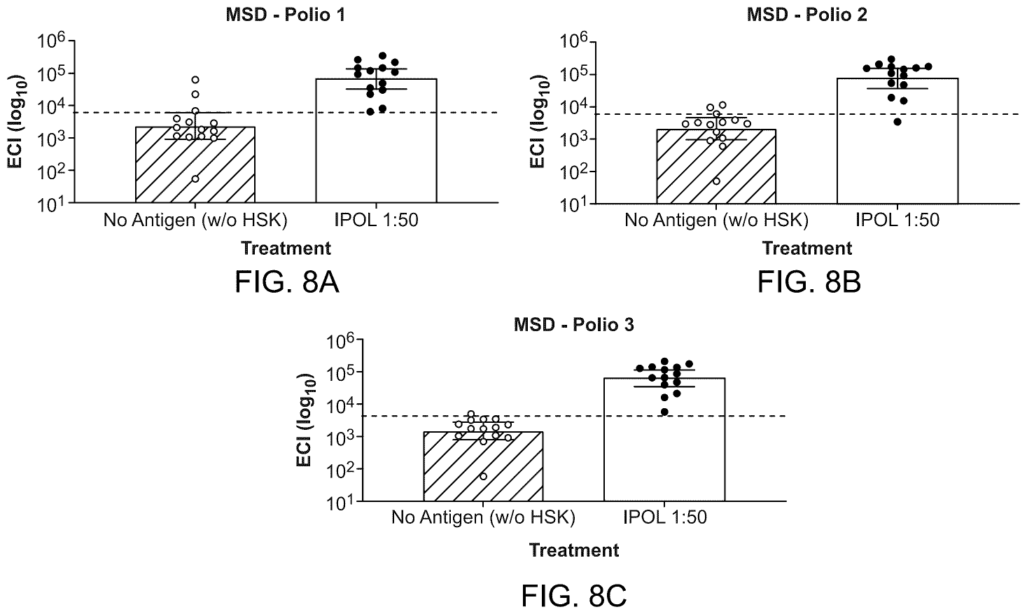

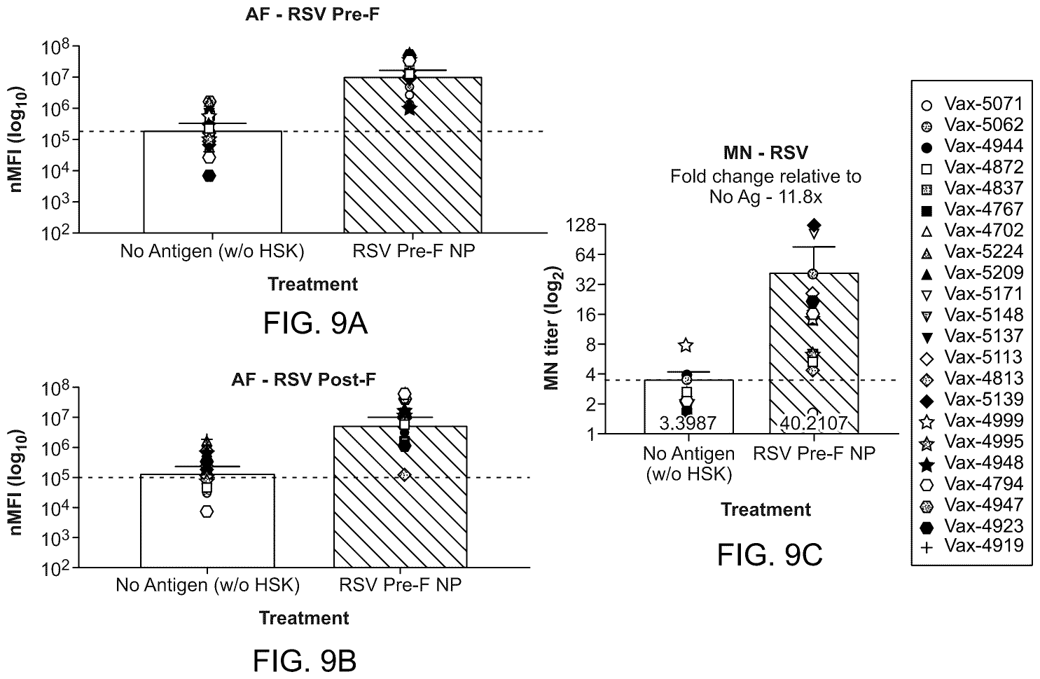

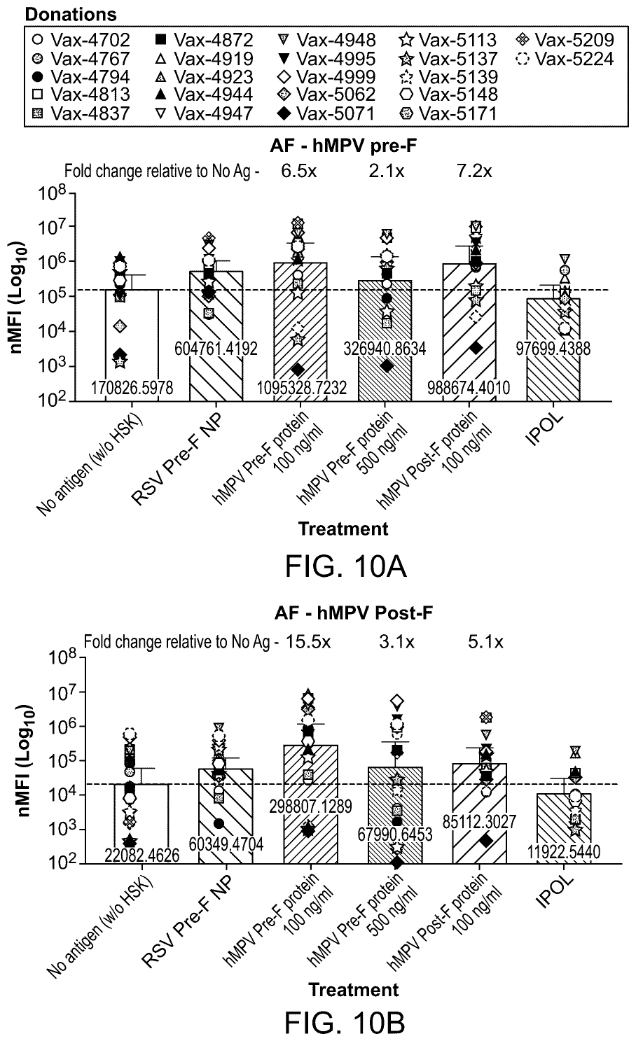

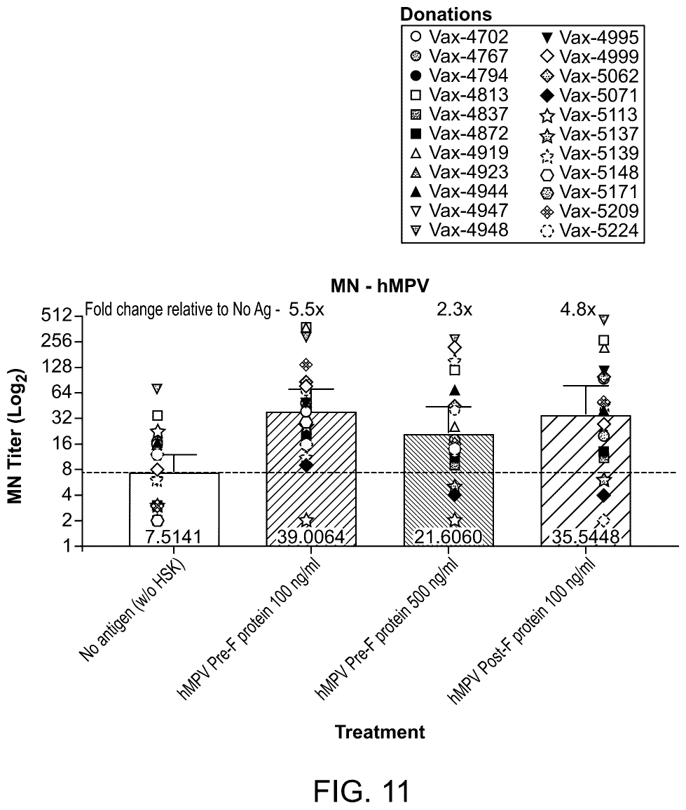

To confirm the activation MIMIC co-cultures, previously analyzed polio vaccine (IPOL) and antigen (RSV pre-F-NP) were used as positive controls. As shown in FIGS. 8A-8C, the IPOL treatment in a 1:50 dilution elicited an antibody response to three Polio strains (Polio 1, 2, and 3) relative to untreated control. Similarly, 50 ng/ml RSV Pre-F NP treatment of co-culture elicited an IgG-specific antibody response to both RSV Pre-F (FIG. 9A) and RSV Post-F (FIG. 9B). Further, these antibodies were also functional as measured in an RSV neutralization assay (FIG. 9C). Supernatants from cocultures treated with experimental groups, hMPV pre-F antigen protein (at 100 ng/ml or 500 ng/ml) or hMPV post F antigen protein (100 ng/ml) elicited a robust IgG antibody response to both hMPV pre-F (FIG. 10A) and hMPV post-F antigen (FIG. 10B) relative to no antigen control. Also, these antibodies were functional as measured in a hMPV neutralization assay (FIG. 11). Antibodies from all three treatment groups bound to hMPV pre- and post-fusion F antigen and neutralized viral infectivity supporting the notion that pre- and post-fusion hMPV share neutralizing epitopes.

### Example 7: Evaluation of Protein Expression from Optimized Panel of rB/HPIV3-hMPV F Constructs

### Introduction

The expression of the vector proteins and HMPV F from the B/HPIV3 vectors expressing the eight different HMPV (CAN97-83) F versions designed in Example 1 were assessed. Vero cells were infected at a multiplicity of infection (MOI) of 3 PFU per cell with the eight B/HPIV3/HMPV constructs, the empty B/HPIV3 vector, wild-type HMPV CAN97-83, or were mock-infected. The cells were incubated for 48 hours at 32° C., and cell lysates were prepared and analyzed by Western blotting.

### Material & Methods:

Cells and Viruses. Cells were cultures and virus was propagated as described in Example 1.

Analysis of viral protein expression by Western blotting. The expression of PIV3 vector proteins and HMPV F protein was evaluated. Vero cells were infected at an MOI of 3 PFU/cell with the indicated viruses and incubated for 48 hours at 32° C. followed by cell lysis, SDS PAGE (4-12% Bis-tris gels under reducing and denaturing conditions), and Western blotting as previously described. Liu et al. (2020), PLoS One 15: e0228572. HMPV F protein was detected using a hamster monoclonal antibody (mAb 1017). The corresponding species-specific infra-red dye conjugated secondary antibodies were used to visualize proteins on an infra-red scanner as described above.

### Results:

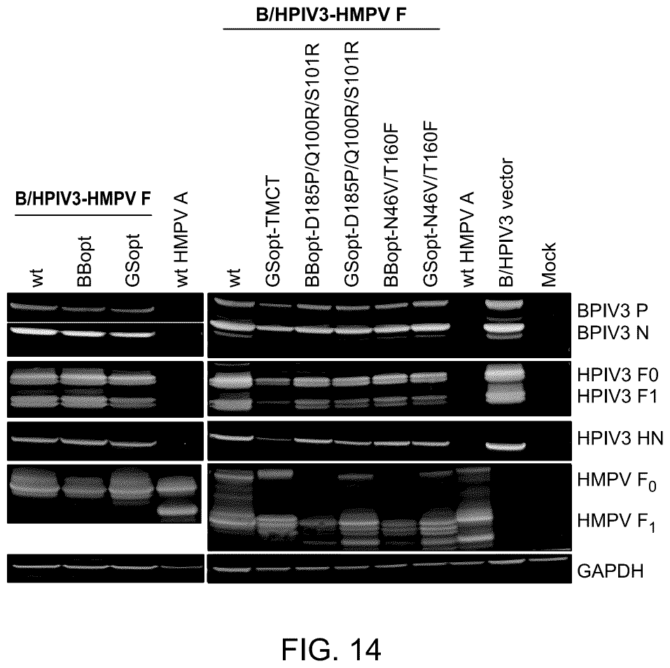

As shown in FIG. 14, all eight HMPV F versions were efficiently expressed by the B/HPIV3 vector and both the F0 precursor and the F1 protein subunit were detected using a monoclonal antibody specific to HMPV F. An increase in expression of HMPV F was not observed for BBopt or GSopt HMPV F compared to wild-type HMPV F. Specifically, expression of HMPV F by the GSopt construct was similar to that of wild-type HMPV F whereas expression of HMPV F by the BBopt construct was somewhat reduced compared to wild-type and GSopt. Thus, contrary to expectations, the codon optimization did not increase expression and indeed the BBopt optimization unpredictably decreased expression. The other constructs based on GSopt (GSopt-D185P/Q100R/S101R, GSopt-N46V/T160F) also had levels of F expression similar to wild-type HMPV, whereas the other constructs based on BBopt (BBopt-D185P/Q100R/S101R, BBopt-N46V/T160F) had reduced levels of F expression.

The amounts of vector expressed BPIV3 N and P proteins and HPIV3 F and HN proteins were reduced for all viruses expressing HMPV F as compared to the empty B/HPIV3 control. The reduction was modest for N protein but was greater for the P, F, and HN proteins. This likely reflects the placement of the HMPV F gene in the second gene position immediately following the N gene. In this location, due to 3′-5′ gradient of virus transcription, the insert might have minimal effect on expression of the upstream N gene but could cause greater reduction in the expression of the other viral genes located downstream. This might be the basis for the reduced plaque sizes associated with insertion of the HMPV F gene. The GSopt-TMCT construct had a greater reduction in the global expression of the PIV3 proteins compared to the other constructs, which was consistent with, and might be the basis for, its greatly reduced plaque size as compared to other viruses. Despite this, its level of expression of the HMPV F protein was comparable to the other GSopt versions.

### Example 8: Immunoqenicity of Optimized Panel of rB/HPIV3-hMPV F Constructs in Hamsters

### Introduction

The immunogenicity and efficacy of the B/HPIV3 vectors expressing the eight different HMPV (CAN97-83) F versions designed in Example 1 was tested.

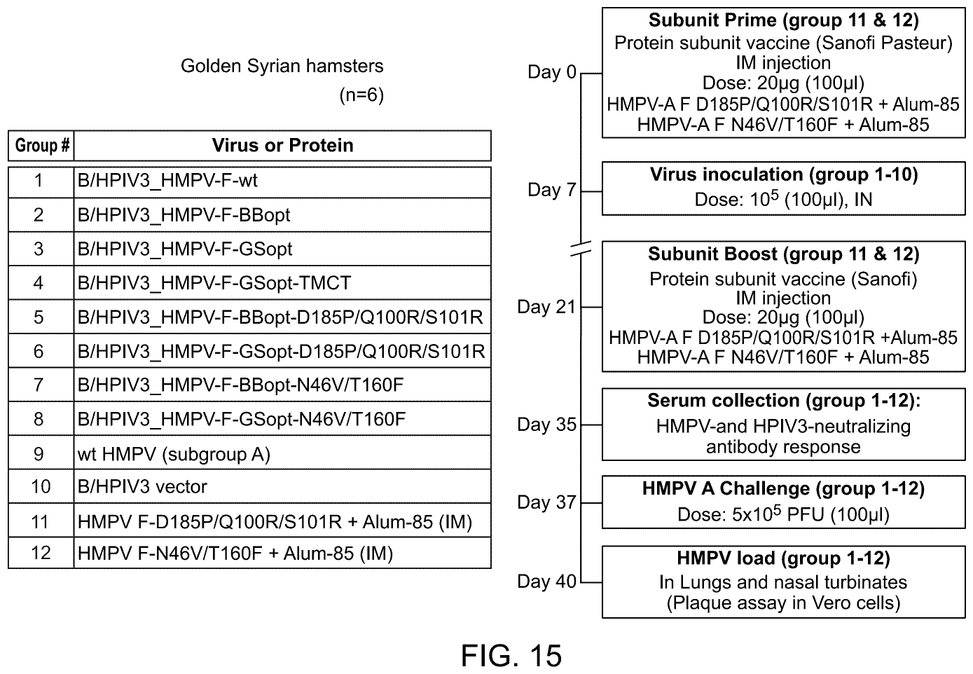

The experimental design is shown in FIG. 15. Briefly, groups of 6 hamsters each were immunized with one of the eight viral B/HPIV3-hMPV F constructs, wild-type HMPV, or an empty vector control at a single intranasal dose, or with either of the two purified HMPV F subunit recombinant proteins (F-D185P/Q100R/S101R and F-N46V/T160F) intramuscularly at two doses three weeks apart. Serum samples were collected from all animals at 4 weeks post virus immunization or 2 weeks post second intramuscular protein dose and the serum HMPV- and HPIV3-neutralizing antibody titers were determined by 60% plaque reduction neutralization assays.

### Material & Methods:

Animal study protocols were approved by the NIH Animal Care and Use Committee. Six-week-old Golden Syrian hamsters, in groups of 6, were inoculated intranasally with a 10⁵ PFU dose of rB/HPIV3-HMPV F vector administered as a single dose. The B/HPIV3 vector without HMPV F insert and wt HMPV (subgroup A, strain CAN97-83, GenBank accession: AY297749) were included as controls. Two groups of 6 hamsters each were immunized by intramuscular administration of 20 μg per dose of either of two purified HMPV F proteins, F-D185P/Q100R/S101R or F-N46V/T160F, mixed 1:1 (vol:vol) with Alum-85 adjuvant as two doses administered 3 weeks apart. Serum samples were collected from immunized hamsters at 4 weeks post virus immunization or 2 weeks post second IM protein dose. Two days after serum collection, all hamsters were challenged intranasally with 5×10⁵ PFU of wt HMPV (CAN97-83). At day 3 post-challenge, animals were euthanized, and nasal turbinates and lungs were collected to quantify HMPV replication in those tissues. Tissue homogenates were prepared and titrated by HMPV plaque assay on Vero cells with data reported as PFU/g of each tissue.

The HMPV- and HPIV3-specific 60% plaque reduction neutralization titers (PRNT60) of hamster sera were determined as previously described using wild-type HMPV strain CAN97-83 and HPIV3 that expresses green fluorescent protein, respectively, and were reported as Log 2 PRNT60. Liu et al. (2020), PLoS One 15:e0228572; Skiadopoulos et al. (2004), J Virol 78:6927-37.; Bernstein et al. (2012), Infect Dis J 31:109-14.

### Results:

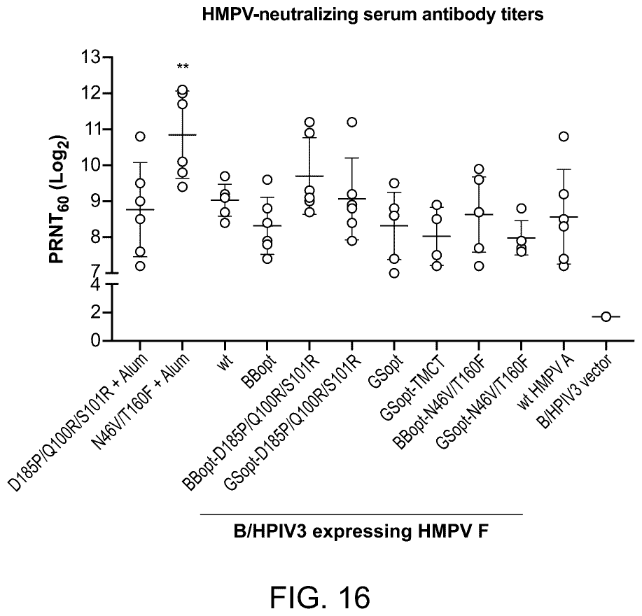

As shown in FIG. 16, all B/HPIV3 vector constructs expressing HMPV F induced serum HMPV-neutralizing antibody titers that were comparable to those induced by wild-type HMPV A with no statistically significant differences. The subunit protein vaccines also induced serum HMPV-neutralizing antibodies; the F-N46V/T160F conferred significantly higher titers (p<0.01) while those of F-D185P/Q100R/S101R were similar to wild-type HMPV A control.

On day 37 post vaccine administration, to assess the level of protection against infection, all hamsters were challenged with an intranasal dose of 5×10⁵ PFU of wild-type HMPV A (CAN97-83). On day 3 post-challenge, nasal turbinates and lungs were collected and the load of challenge HMPV was quantified by HMPV plaque assay.

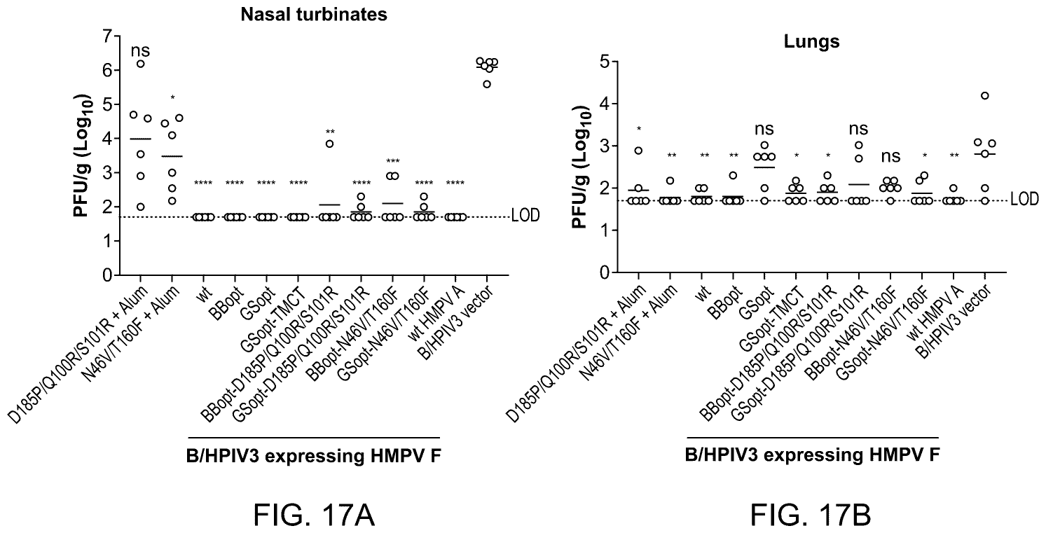

In the nasal turbinates, wild-type HMPV replicated to high titers in the B/HPIV3 vector (empty) immunized animals (mean: 10⁶ PFU/g). (FIG. 17A) while all the rB/HPIV3 vectors constructs expressing different forms of HMPV F were highly protective with either none or significantly reduced HMPV replication. The wt HMPV A immunized control group was completely protected with no detectable infectious virus. B/HPIV3 vector constructs expressing the native forms of HMPV F protein, i.e., wild-type HMPVA, BBopt, and GSopt, were the most protective and had no detectable infectious HMPV in the nasal turbinates (p<0.0001). Similarly, GSopt-TMCT also provided complete protection with no infectious virus detected in the nose (p<0.0001). Vectors expressing the BBopt or GSopt versions with pre-F stabilizing D185P/Q100R/S101R or N46V/T160F mutations also showed significantly reduced HMPV titers with means ranging from 10^{1.8} to 10^{2.1} PFU/g (P<0.0001 to p<0.001). Each of the B/HPIV3 vector constructs expressing HMPV F had majority of animals with no detectable virus with only one or two animals mostly showing very low HMPV replication.

In the lung homogenates, as shown in FIG. 17B, the results were less clear in the lungs due to an overall low replication of the HMPV challenge virus in all groups including the HMPV naïve (B/HPIV3 vector) group. The GSopt, GSopt-D185P/Q100R/S101R, and BBopt-N46V/T160F viruses did not provide significant protection in the lungs. The remaining five rB/HPIV3 vectors expressing HMPV F conferred significant protection in the lungs with mean HMPV challenge virus titers ranging from 10^{1.8} to 10^{1.9} PFU/g (p<0.01 to p<0.05). Both of the purified protein vaccines F-D185P/Q100R/S101R and F-N46V/T160F also showed significant protection and reduced HMPV mean titers to 10^{1.94} (P<0.05) and 10^{1.77} (p<0.01) PFU/g, respectively, indicating that the high serum HMPV-neutralizing antibody titers induced by them were protective in the lungs.

In summary, the hamster study showed that the B/HPIV3 vectors expressing the various forms of HMPV F were at least as immunogenic as infection with wild-type HMPV. Immunization with these B/HPIV3 vectors was as protective as wild-type HMPV in the upper respiratory tract. Furthermore, a single intranasal dose of any of the vectors was more protective in the upper respiratory tract than were two large, adjuvanted intramuscular doses of the corresponding purified pre-F HMPV F proteins (compare FIGS. 17A-17B). The similarity or superiority of the vectors versus the purified protein with regard to serum HMPV-neutralizing titers and protective efficacy was remarkable because (i) the protein vaccines were given with adjuvant, and (ii) the protein vaccines were given in two doses whereas the vectors were given in single dose, and secondary immune responses typically are much higher and more protective than primary responses. The superior protection in the upper respiratory tract by the vectors likely due to direct stimulation of the respiratory tract immunity, indicating the importance of the intranasal route of administration. In the lower respiratory tract, the level of challenge HMPV replication was generally similar in all of the immunized animals. This assessment was confounded by the generally low replication of challenge HMPV in the lower respiratory tract. Nonetheless, all of the immunogens appeared to confer approximately comparable levels of protection in the lower respiratory tract.

Thus, the B/HPIV3 vector, which previously has been shown to be satisfactorily attenuated and safe in young children, can express HMPV F protein to provide an immunogenic, protective, and stable vaccine. Given the previous demonstration of safety of the B/HPIV3 and B/HPIV3/RSV vectors in young children, these HMPV-F-expressing derivatives are suitable to be evaluated directly in young children as an intranasal vaccine against HPIV3 and HMPV that would be superior to two intramuscular doses of purified protein.

Other embodiments of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

All patents and publications cited herein are incorporated by reference herein in their entirety.

1. A viral vector that encodes a human metapneumovirus (hMPV) F polypeptide antigen that lacks a transmembrane domain, lacks a cytoplasmic tail, and comprises a human rhinovirus 3C (HRV-3C) protease cleavage site.

2. The viral vector of claim 1, comprising a viral vector backbone derived from a parainfluenza virus (PIV), optionally wherein the PIV is a chimeric bovine/human parainfluenza type 3 virus (rB/HPIV3) or a human parainfluenza type 3 virus (HPIV3).

3. The viral vector of claim 1, wherein:
said F polypeptide further comprises an F₀ cleavage site mutation comprising amino acid substitutions Q100R and S101R, replacing glutamine at amino acid position 100 of SEQ ID NO: 1 with arginine, and replacing serine at amino acid position 101 of SEQ ID NO: 1 with arginine,
said F polypeptide further comprises a signal peptide;
said F polypeptide further comprises at least one tag sequence that is optionally an 8×His tag and/or a Strep II tag; and/or
said F polypeptide further comprises a foldon domain; and/or
the hMPV F polypeptide is a pre-fusion F polypeptide.

4-6. (canceled)

7. The viral vector of claim 1, wherein the F polypeptide further comprises:
an amino acid substitution replacing the amino acid at position 160 of SEQ ID NO: 1, and an amino acid substitution replacing the amino acid position 46 of SEQ ID NO: 1;
an amino acid substitution replacing the amino acid at position 160 with phenylalanine, tryptophan, tyrosine, valine, alanine, isoleucine, or leucine;
an amino acid substitution replacing the amino acid position 160 with phenylalanine;
an amino acid substitution replacing the amino acid position 46 with valine, alanine, isoleucine, leucine, phenylalanine, tyrosine, or proline;
an amino acid substitution replacing the amino acid position 46 with valine;
an amino acid substitution replacing the amino acid at position 160 of SEQ ID NO: 1 with phenylalanine, tryptophan, tyrosine, valine, alanine, isoleucine, or leucine and/or an amino acid substitution replacing the amino acid position 46 of SEQ ID NO: 1 with valine, alanine, isoleucine, leucine, phenylalanine, tyrosine, or proline; and/or
an amino acid substitution replacing threonine at amino acid position 160 of SEQ ID NO: 1, and an amino acid substitution replacing asparagine at amino acid position 46 of SEQ ID NO: 1.

8-13. (canceled)

14. The viral vector of claim 1, wherein the viral vector encodes a hMPV F polypeptide antigen,
wherein said F polypeptide lacks a transmembrane domain and lacks a cytoplasmic tail, and further comprises:
an F₀ cleavage site mutation comprising amino acid substitutions Q100R and S101R; replacing glutamine at amino acid position 100 of SEQ ID NO: 1 with arginine, and replacing serine at amino acid position 101 of SEQ ID NO: 1 with arginine;
a HRV-3C protease cleavage site;
a heterologous signal peptide;
an 8×His tag and/or a Strep II tag; and
a foldon domain.

15. A viral vector that encodes an hMPV F polypeptide antigen, wherein said F polypeptide lacks a transmembrane domain, lacks a cytoplasmic tail, and comprises an amino acid substitution replacing threonine at amino acid position 160 of SEQ ID NO: 1, and an amino acid substitution replacing asparagine at amino acid position 46 of SEQ ID NO: 1.

16. The viral vector of claim 15, wherein said F polypeptide comprises:
an amino acid substitution replacing threonine at amino acid position 160 with phenylalanine, tryptophan, tyrosine, valine, alanine, isoleucine, or leucine;
an amino acid substitution T160F replacing threonine at amino acid position 160 with phenylalanine;
an amino acid substitution replacing asparagine at amino acid position 46 with valine, alanine, isoleucine, leucine, phenylalanine, tyrosine, or proline;
an amino acid substitution N46V replacing asparagine at amino acid position 46 with valine;
at least 95% sequence identity to SEQ ID NO: 7;
an F₀ cleavage site mutation comprising amino acid substitutions Q100R and S101R, replacing glutamine at amino acid position 100 of SEQ ID NO: 1 with arginine, and replacing serine at amino acid position 101 of SEQ ID NO: 1 with arginine;
a signal peptide;
at least one tag sequence that is optionally an 8×His tag and/or a Strep II tag;
a foldon domain;
optionally wherein the hMPV F polypeptide is a pre-fusion F polypeptide; and/or
the viral vector further comprising a hMPV F nucleic acid molecule having at least 95% sequence identity to SEQ ID NO: 8, optionally wherein the nucleic acid molecule comprises SEQ ID NO: 8.

17-24. (canceled)

25. The viral vector of claim 15, that encodes an antigenic hMPV prefusion F polypeptide, wherein said prefusion F polypeptide lacks a transmembrane domain, lacks a cytoplasmic tail, and comprises:
an amino acid substitution T160F replacing threonine at amino acid position 160 of SEQ ID NO: 1 with phenylalanine, and an amino acid substitution N46V replacing asparagine at amino acid position 46 of SEQ ID NO: 1 with valine;
an F₀ cleavage site mutation comprising amino acid substitutions Q100R and S101R; replacing glutamine at amino acid position 100 of SEQ ID NO: 1 with arginine, and replacing serine at amino acid position 101 of SEQ ID NO: 1 with arginine;
a HRV-3C protease cleavage site;
a signal peptide;
an 8×His tag and/or a Strep II tag; and
a foldon domain.

26. The viral vector of claim 1, wherein
the hMPV F is from A strain hMPV; and/or
the hMPV F is A1 subtype or A2 subtype hMPV.

27. (canceled)

28. The viral vector of claim 1, wherein the F polypeptide comprises at least 95% sequence identity to SEQ ID NO: 3 or comprising SEQ ID NO: 3.

29. A viral vector encoding an hMPV F polypeptide, wherein said F polypeptide comprises at least 95% sequence identity to SEQ ID NO: 7.

30. The viral vector of claim 29, wherein:
the F polypeptide is a prefusion F polypeptide;
the viral vector backbone derived from a (PIV), optionally wherein the PIV is a chimeric rB/HPIV3 or a HPIV3;
the F polypeptide comprises amino acid substitution T160F replacing threonine at amino acid position 160 with phenylalanine, and amino acid substitution N46V replacing asparagine at amino acid position 46 with valine;
the F polypeptide comprises SEQ ID NO: 7; and/or
the viral vector further comprising a hMPV F nucleic acid molecule having at least 95% sequence identity to SEQ ID NO: 8, optionally wherein the nucleic acid molecule comprises SEQ ID NO: 8.

31-38. (canceled)

39. A live-attenuated virus or a pharmaceutical composition comprising the viral vector of claim 1, optionally wherein the live-attenuated virus or the pharmaceutical composition is comprised in a vaccine.

40-41. (canceled)

42. A method of eliciting an immune response to hMPV and/or HPIV3 or protecting a subject against hMPV infection and/or HPIV3 infection, comprising administering the vaccine of claim 39 to a subject.

43. The method of claim 42, wherein:
the vaccine is co-administered with an adjuvant,
the vaccine is administered in combination with an additional vaccine, optionally wherein the additional vaccine is a respiratory syncytial virus (RSV) vaccine or an influenza vaccine;
the subject is human, optionally wherein the human subject is an infant, a toddler, or an older adult; and/or
the vaccine increases the serum concentration of neutralizing antibodies, and wherein the subject has pre-existing hMPV immunity.

44-49. (canceled)

50. The viral vector of claim 1, in the manufacture of a medicament for eliciting an immune response to hMPV and HPIV3 or protecting a subject against hMPV infection and HPIV3 infection.

51. A method of:
(A) eliciting an immune response in a subject in need thereof, comprising administering to the subject, optionally intramuscularly, intranasally, intravenously, subcutaneously, or intradermally, a prophylactically effective amount of the viral vector of claim 1; or
(B) preventing an hMPV infection and an HPIV3 infection or reducing one or more symptoms of an hMPV infection and an HPIV3 infection, comprising administering to the subject, optionally intramuscularly, intranasally, intravenously, subcutaneously, or intradermally, a prophylactically effective amount of the viral vector of claim 1.

52-53. (canceled)

54. The viral vector of a claim 1, for use in treating a subject in need thereof.

55. A kit comprising a container comprising a single-use or multi-use dosage of the viral vector of claim 1, optionally wherein the container is a vial or a pre-filled syringe or injector.

56. The viral vector of a claim 1, wherein the viral vector comprises a hMPV F nucleic acid molecule having at least 95% sequence identity to SEQ ID NO: 8, optionally wherein the nucleic acid molecule comprises SEQ ID NO: 8.
